(19) United States
(12) Patent Application Publication
(10) Pub. No.: US 2023/0059132 A1
(43) Pub. Date: Feb. 23, 2023

(54) SYSTEM AND METHOD FOR DEEP LEARNING FOR INVERSE PROBLEMS WITHOUT TRAINING DATA

(71) Applicant: The General Hospital Corporation, Boston, MA (US)

(72) Inventors: Quanzheng Li, Cambridge, MA (US); Kuang Gong, Malden, MA (US)

(21) Appl. No.: 17/764,459

(22) PCT Filed: Sep. 28, 2020
(86) PCT No.: PCT/US2020/053166

§ 371 Date: Mar. 28, 2022

Related U.S. Application Data
(60) Provisional application No. 62/906,597, filed on Sep. 26, 2019.

(51) Int. Cl. G06N 3/08; G16H 30/20

(52) U.S. Cl. CPC G06N 3/08; G16H 30/20

(57) ABSTRACT

In accordance with one aspect of the disclosure, an image generation system is provided. The system includes at least one processor and at least one non-transitory, computer- readable memory accessible by the processor and having instructions that, when executed by the processor, cause the processor to receive a first patient image associated with a patient, receive a second patient image associated with the patient, train an untrained model based on the first patient image and the second patient image, provide the first patient image to the model, receive a third patient image from the model, and output the third patient image to at least one of a storage system or a display.

### CROSS-REFERENCE TO RELATED APPLICATIONS

This application is based on, claims the benefit of, and claims priority to, U.S. Provisional Patent Application No. 62/906,597, filed Sep. 26, 2019, which is hereby incorporated herein by reference in its entirety for all purposes.

### STATEMENT REGARDING FEDERALLY SPONSORED RESEARCH

The invention was made with government support under R01AG052653, P41EB022544, C06CA059267, P41EB022544, T32EB013180, R01HL118261, R01HL137230, and grant R01CA165221 awarded by the National Institutes of Health. The government has certain rights in this invention.

### BACKGROUND

Medical imaging is a valuable tool in the practice of modern clinical medicine. Imaging is used in an extremely broad array of clinical situations, from diagnosis to delivery of therapeutics to guiding surgical procedures. Machine learning techniques requiring training data have been used for various tasks with medical imaging. However, these techniques require large amounts of data sets. For medical imaging tasks such as lesion detection and region-of-interest (ROI) quantification, obtaining high quality diagnostic images is essential. Recently the neural network method has been applied to transform low-quality images into the images with improved signal-to-noise ratio (SNR). In some cases collecting large amounts of training labels is relatively easy, such as static magnetic resonance (MR) reconstruction. However, this is not an easy task for some other cases.

High-dose computed tomography (CT) has potential safety concerns; long-scanned dynamic positron emission tomography (PET) is not employed in routine clinical practice; in cardiac MR applications, it is practically impossible to acquire breathhold fully sampled 3D images. With limited amounts of high-quality patient data sets available, overfitting can be a potential pitfall. For example, if a new patient data does not lie in the training space due to population difference, the trained network cannot accurately recover unseen structures. In addition, low-quality images are often simulated by artificially downsampling the full-dose/ high-count data, which may not reflect the real physical condition of low-dose imaging. This mismatch between training and the real clinical environment can reduce the network performance.

Thus, systems and methods that overcome the drawbacks of present machine learning-based medical imaging analysis techniques that require large amounts of training data are desired.

### SUMMARY OF THE DISCLOSURE

The present disclosure provides systems and methods that improve image reconstruction and denoising without the use of training data. In one non-limiting example, systems and methods are provided for reconstructing and/or denoising images, for example, such as positron emission tomography (PET) images.

In accordance with one aspect of the disclosure, an image generation system is provided. The system includes at least one processor and at least one non-transitory, computer-readable memory accessible by the processor and having instructions that, when executed by the processor, cause the processor to receive a first patient image associated with a patient, receive a second patient image associated with the patient, train an untrained model based on the first patient image and the second patient image, provide the first patient image to the model, receive a third patient image from the model, and output the third patient image to at least one of a storage system or a display.

In accordance with another aspect of the disclosure, an image generation method is provided. The method includes receiving a first patient image associated with a patient, receiving a second patient image associated with the patient, training an untrained model based on the first patient image and the second patient image, providing the first patient image to the model, receiving a third patient image from the model, and outputting the third patient image to at least one of a storage system or a display.

The foregoing and other aspects and advantages of the invention will appear from the following description. In the description, reference is made to the accompanying drawings which form a part hereof, and in which there is shown by way of illustration configurations of the invention. Any such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an example of an image generation system in accordance with the disclosed subject matter.

FIG. 2 is a block diagram of an example of hardware that can be used to implement a computing device and a supplemental computing device shown in FIG. 1 in accordance with the disclosed subject matter.

FIG. 3 is a block diagram of a positron emission tomography (PET) system that includes a detector system having a detector ring assembly in accordance with the disclosed subject matter.

FIG. 4 is a flowchart setting forth some example steps for generating a reconstructed image in accordance with the disclosed subject matter.

FIG. 5 is a schematic diagram of an exemplary neural network for use in a deep learning framework in accordance with the present disclosure.

FIG. 6 is a flow chart setting for steps of an example method for training a model in accordance with the present disclosure.

FIG. 7A is a patient image generated using deep image prior (DIP) framework with random noise as network input.

FIG. 7B is a patient image generated using conditional DIP framework with a magnetic resonance (MR) image of the patient as network input.

FIG. 7C is a patient image generated using the neural network trained in accordance with FIG. 6 and using an MR image of the patient as network input.

FIG. 8 is a plot of normalized cost value curves for different algorithms.

FIG. 9A is a plot of contrast recovery coefficient (CRC) vs. standard deviation (STD) curves at the gray matter region in the simulation dataset.

FIG. 9B is a plot of CRC-STD curves at the gray matter region in the simulation dataset.

FIG. 10 is a plot of CR-STD curves for different methods.

FIG. 11 is a flow chart setting forth some example steps of another exemplary process for training a model in accordance with the present disclosure.

FIG. 12 is a flow chart for generating and/or reconstructing Arterial Spin Labeling (ASL) MR images in accordance with the present disclosure.

FIG. 13 is a schematic diagram of yet another exemplary neural network in accordance with the present disclosure.

FIG. 14 is a flow chart setting forth some example steps of yet another exemplary process for training a model in accordance with the present disclosure.

FIG. 15 is a schematic diagram of still yet another exemplary neural network in accordance with the present disclosure.

FIG. 16 is a flow chart setting forth some example steps of still another exemplary process for training a model in accordance with the present disclosure.

FIG. 17 is a flow chart setting forth some example steps of an additional exemplary process for training a model in accordance with the present disclosure.

### DETAILED DESCRIPTION

The present disclosure provides systems and methods that can generate certain types of images (e.g., reconstructed images, denoised images, and or images of a various imaging modalities) using certain imaging modalities without training data. In particular, the present disclosure provides systems and method for generating new images such as reconstructed positron emission tomography (PET) images, denoised PET images, arterial spin labeling (ASL) images, low-dose dual energy computed tomography (DECT) images, and PET parametric images associated with a patient using combinations of certain medical images such as PET images, ASL images, CT images, MR images, and/or DECT images of the patient without training data. As one non-limiting example, a system and method is provided for generating a reconstructed PET image of a patient using a PET image of the patient and a simultaneously acquired MR image of the patient.

Previous techniques for reconstructing and/or denoising medical images generally have one or more drawbacks. As described above, machine learning approaches that rely on training data are prone to overfitting due to limited amounts of high-quality patient data sets available. Apart from using training pairs to perform supervised learning, some techniques use prior images acquired from the same patient to improve the image quality. The prior images can come from temporal information, different physics settings, or even other imaging modalities. They are included into the maximum posterior estimation or sparse representation framework using predefined analytical expressions or pre-learning steps. The predefined expressions might not be able to extract all the useful information in a given image, and the pre-learnt model might not be optimal for the later reconstruction as no data consistency constraint is enforced during pre-learning. Other techniques use neural networks to generate various distributions based on random noise input. However, random noise may not provide enough information to sufficiently reconstruct and/or denoise an image to a desired level.

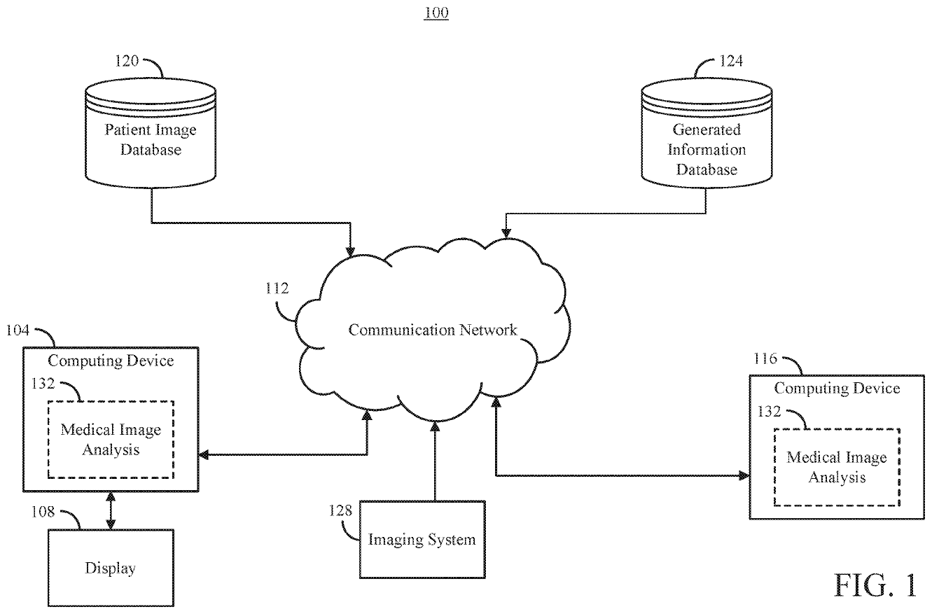

The present disclosure provides systems and methods that overcome at least one of the drawbacks of the above techniques. FIG. 1 shows an example of an image generation system 100 in accordance with some aspects of the disclosed subject matter. In some configurations, the image generation system 100 can include a computing device 104, a display 108, a communication network 112, a supplemental computing device 116, a patient image database 120, a generated information database 124, and/or an imaging system 128. In some configurations, the computing device 104 can be in communication (e.g., wired communication, wireless communication) with the display 108, the supplemental computing device 116, the patient image database 120, the generated information database 124, and/or the imaging system 128.

The computing device 104 can implement portions of a medical image analysis application 132, which can involve the computing device 104 transmitting and/or receiving instructions, data, commands, etc. from one or more other devices. For example, the computing device 104 can receive patient image data (e.g., PET images, ASL images, CT images, MR images, and/or DECT images) from the patient image database 120, receive patient image data (e.g., PET images, ASL images, CT images, MR images, and/or DECT images) from the imaging system 128, and/or transmit reports, raw data, and/or other information generated by the medical image analysis application 132 to the display 108 and/or the generated information database 124.

The supplementary computing device 116 can implement portions of the medical image analysis application 132. It is understood that the image generation system 100 can implement the medical image analysis application 132 without the supplemental computing device 140. In some aspects, the computing device 104 can cause the supplemental computing device 116 to receive patient image data (e.g., PET images, ASL images, CT images, MR images, and/or DECT images) from the patient image database 120, receive patient image data (e.g., PET images, ASL images, CT images, MR images, and/or DECT images) from the imaging system 128, and/or transmit reports, raw data, and/or other information generated by the medical image analysis application 132 to the display 108 and/or the generated information database 124. In this way, a majority of the medical image analysis application 132 can be implemented by the supplementary computing device 116, which can allow a larger range of devices to be used as the computing device 104 because the required processing power of the computing device 104 may be reduced. For example, a relatively inexpensive tablet computer or laptop computer may be used as the computing device 104, and a server may be used as the supplementary computing device 116.

The patient image database can include patient image data previously generated by an imaging device (e.g., the imaging system 128). In some configurations, the imaging system 128 can be configured to generate PET images, ASL images, CT images, MR images, and/or DECT images. In some configurations, the imaging system 128 can include a single imaging device configured to produce multiple types of images simultaneously and/or concurrently. For example, the imaging system 128 can include a PET scanner that can also generate a CT scan concurrently. In this way, a single imaging machine can supply sufficient input data to the medical image analysis application 132 in order to generate certain types of images (e.g., a reconstructed PET image and/or a denoised PET image). In some configurations, the imaging system 128 can include multiple devices each configured to generate a single type of image (e.g., a first device configured to generate PET images and a second device configured to generate MR images).

The generated information database 124 can include patient image data generated by the medical image analysis application 132. The patient image data may be formatted in the DICOM® standard (i.e., format) (e.g., as a DICOM® object). Each DICOM® object can include image data (e.g., pixel data) formatted in various standards such as JPEG, lossless JPEG, JPEG 2000, etc. Each DICOM® object can also include attributes about the patient and/or the image data (imaging modality and/or image type).

In some configurations, medical image analysis application 132 can automatically generate one or more images (e.g., PET images, ASL images, CT images, MR images, and/or DECT images) based on patient images without training data. The medical image analysis application 132 can also automatically generate one or more reports based on the generated images. The medical image analysis application 132 can output one or more of the generated images and/or reports to the display 108 (e.g., in order to display the generated image and/or report to a medical practitioner) and/or to a memory, such as a memory included in the generated information database 124 (e.g., in order to store the generated image and/or report).

As shown in FIG. 1, the communication network 112 can facilitate communication between the computing device 104, the supplemental computing device 116, the patient image database 120, and/or the generated information database 124. In some configurations, communication network 112 can be any suitable communication network or combination of communication networks. For example, communication network 112 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CD MA, GSM, LTE, LTE Advanced, WiMAX, etc.), a wired network, etc. In some configurations, communication network 112 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and the like.

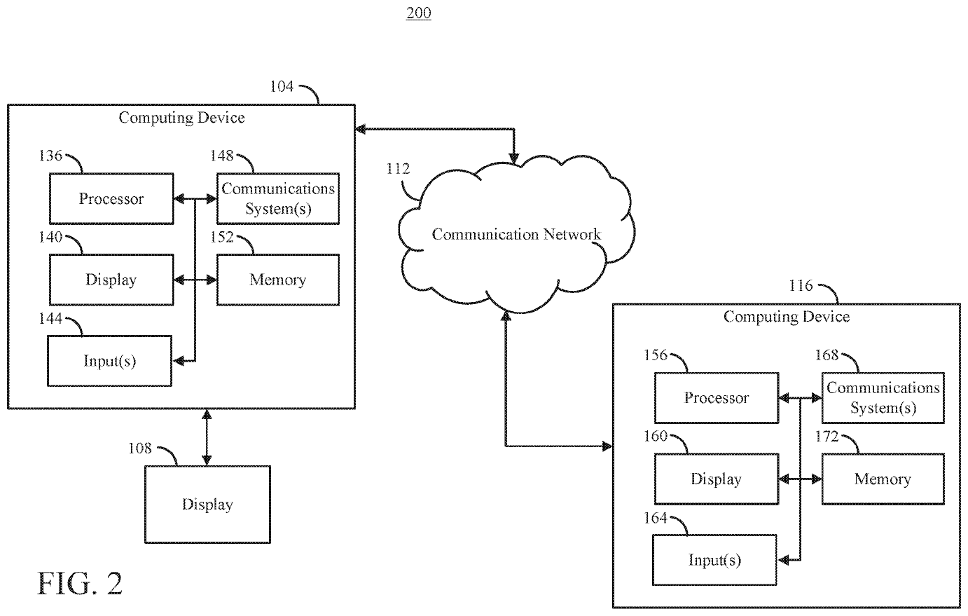

FIG. 2 shows an example of hardware that can be used to implement a computing device 104 and a supplemental computing device 116 shown in FIG. 1 in accordance with some aspects of the disclosed subject matter. As shown in FIG. 2, the computing device 104 can include a processor 136, a display 140, an input 144, a communication system 148, and a memory 152. The processor 136 can implement at least a portion of the medical image analysis application 132, which can, for example, be executed from a program (e.g., saved and retrieved from the memory 152). The processor 136 can be any suitable hardware processor or combination of processors, such as a central processing unit (“CPU”), a graphics processing unit (“GPU”), etc., which can execute a program, which can include the processes described below.

In some configurations, the display 140 can present a graphical user interface. In some configurations, the display 140 can be implemented using any suitable display devices, such as a computer monitor, a touchscreen, a television, etc. In some configurations, the inputs 144 of the computing device 104 can include indicators, sensors, actuatable buttons, a keyboard, a mouse, a graphical user interface, a touch-screen display, etc. In some configurations, the inputs 144 can allow a user (e.g., a medical practitioner, such as an oncologist) to interact with the computing device 104, and thereby to interact with the supplemental computing device 116 (e.g., via the communication network 112). The display 108 can be a display device such as a computer monitor, a touchscreen, a television, and the like.

In some configurations, the communication system 148 can include any suitable hardware, firmware, and/or software for communicating with the other systems, over any suitable communication networks. For example, the communication system 148 can include one or more transceivers, one or more communication chips and/or chip sets, etc. In a more particular example, the communication system 148 can include hardware, firmware, and/or software that can be used to establish a coaxial connection, a fiber optic connection, an Ethernet connection, a USB connection, a Wi-Fi connection, a Bluetooth connection, a cellular connection, etc. In some configurations, the communication system 148 allows the computing device 104 to communicate with the supplemental computing device 116 (e.g., directly, or indirectly such as via the communication network 112).

In some configurations, the memory 152 can include any suitable storage device or devices that can be used to store instructions, values, etc., that can be used, for example, by the processor 136 to present content using the display 140 and/or the display 108, to communicate with the supplemental computing device 116 via communications system(s) 148, etc. The memory 152 can include any suitable volatile memory, non-volatile memory, storage, or any suitable combination thereof. For example, the memory 152 can include RAM, ROM, EEPROM, one or more flash drives, one or more hard disks, one or more solid state drives, one or more optical drives, etc. In some configurations, the memory 152 can have encoded thereon a computer program for controlling operation of the computing device 104 (or the supplemental computing device 116). In such configurations, the processor 136 can execute at least a portion of the computer program to present content (e.g., user interfaces, images, graphics, tables, reports, and the like), receive content from the supplemental computing device 116, transmit information to the supplemental computing device 116, and the like.

Still referring to FIG. 2, the supplemental computing device 116 can include a processor 156, a display 160, an input 164, a communication system 168, and a memory 172. The processor 156 can implement at least a portion of the medical image analysis application 132, which can, for example, be executed from a program (e.g., saved and retrieved from the memory 172). The processor 156 can be any suitable hardware processor or combination of processors, such as a central processing unit (“CPU”), a graphics processing unit (“GPU”), and the like, which can execute a program, which can include the processes described below.

In some configurations, the display 160 can present a graphical user interface. In some configurations, the display 160 can be implemented using any suitable display devices, such as a computer monitor, a touchscreen, a television, etc. In some configurations, the inputs 164 of the supplemental computing device 116 can include indicators, sensors, actuatable buttons, a keyboard, a mouse, a graphical user interface, a touch-screen display, etc. In some configurations, the inputs 164 can allow a user (e.g., a medical practitioner, such as an oncologist) to interact with the supplemental computing device 116, and thereby to interact with the computing device 104 (e.g., via the communication network 112).

In some configurations, the communication system 168 can include any suitable hardware, firmware, and/or software for communicating with the other systems, over any suitable communication networks. For example, the communication system 168 can include one or more transceivers, one or more communication chips and/or chip sets, etc. In a more particular example, the communication system 168 can include hardware, firmware, and/or software that can be used to establish a coaxial connection, a fiber optic connection, an Ethernet connection, a USB connection, a Wi-Fi connection, a Bluetooth connection, a cellular connection, and the like. In some configurations, the communication system 168 allows the supplemental computing device 116 to communicate with the computing device 104 (e.g., directly, or indirectly such as via the communication network 112).

In some configurations, the memory 172 can include any suitable storage device or devices that can be used to store instructions, values, and the like, that can be used, for example, by the processor 156 to present content using the display 160 and/or the display 108, to communicate with the computing device 104 via communications system(s) 168, and the like. The memory 172 can include any suitable volatile memory, non-volatile memory, storage, or any suitable combination thereof. For example, the memory 172 can include RAM, ROM, EEPROM, one or more flash drives, one or more hard disks, one or more solid state drives, one or more optical drives, etc. In some configurations, the memory 172 can have encoded thereon a computer program for controlling operation of the supplemental computing device 116 (or the computing device 104). In such configurations, the processor 156 can execute at least a portion of the computer program to present content (e.g., user interfaces, images, graphics, tables, reports, and the like), receive content from the computing device 104, transmit information to the computing device 104, and the like.

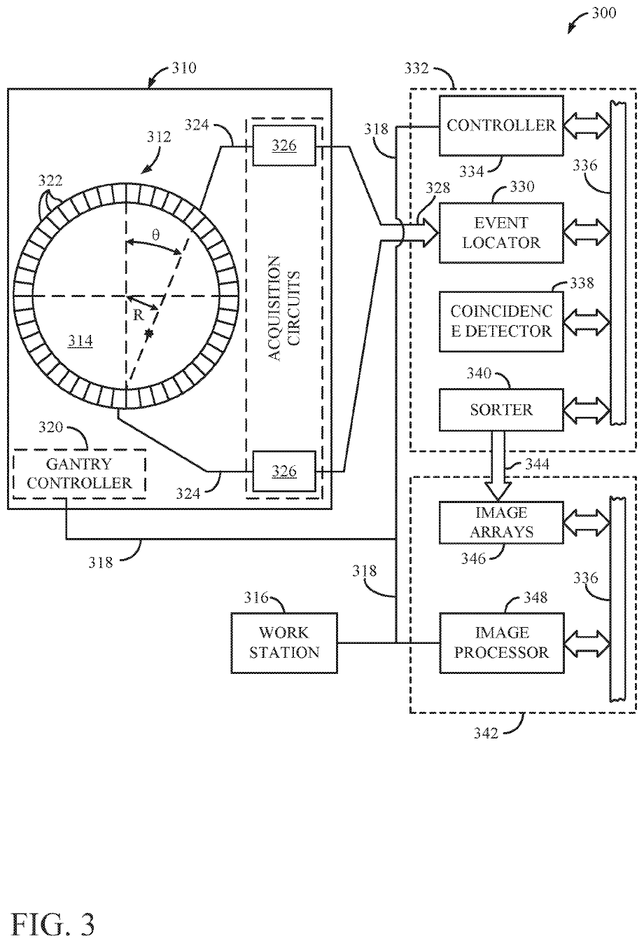

FIG. 3 is a block diagram of a positron emission tomography (PET) imaging system for use with the present disclosure.

Referring now to FIG. 3, a PET system 300 is illustrated that includes a detector system 310 having a detector ring assembly 312. In some configurations, the PET system 300 can be included in the imaging system 128 in FIG. 1. The detector ring assembly 312 is formed of a multitude of radiation detector units 322, represented in this example as block detectors. Each radiation detector unit 322 may include a set of scintillator crystals that is disposed in front of an array of photomultiplier tubes or a position-sensitive photomultiplier tube (not shown), or may be any other suitable radiation detector (for example, such as a high granularity detector). Each radiation detector 322 produces a signal responsive to detection of a photon on communications line 324 when an event occurs. A set of acquisition circuits 326 receive the signals and produce signals indicating the event coordinates (x, y) and the total energy associated with the photons that caused the event. These signals are sent through a cable 328 to an event locator circuit 330. Each acquisition circuit 326 also obtains information from the detector's signals that indicates the exact moment the event took place. For example, with scintillator-type block detectors, digital electronics can obtain this information regarding the precise instant in which the scintillations occurred from the samples of the signals used to obtain energy and event coordinates.

The event locator circuits 330, in some implementations, form part of a data acquisition processing system 332 that processes the signals produced by the acquisition circuits 326. The data acquisition processing system 332 includes a general controller 334 that controls communications, for example, by way of a backplane bus 336 and on the communications network 318. The event locator circuits 330 assemble the information regarding each valid event into a set of numbers that indicate precisely when the event took place, the position in which the event was detected and the energy deposited by the photon. This event data packet is conveyed to a coincidence detector 338 that is also part of the data acquisition processing system 332.

The coincidence detector 338 accepts the event data packets from the event locator circuit 330 and determines if any two of them are in coincidence. The coincidence detector 338 identifies the coincident event pairs located and records them as a coincidence data packet that is provided to a sorter 340. The function of the sorter in many PET imaging systems is to receive the coincidence data packets and generate memory addresses from the coincidence data packets for the efficient storage of the coincidence data. In that context, the set of all projection rays, or lines of response, that point in the same direction (θ) and pass through the scanner's field of view (FOV) is a complete projection, or “view.” The distance (R) between a particular line of response and the center of the FOV locates that line of response within the FOV. The sorter 340 counts all of the events that occur on a given line of response (R, θ) during the scan by sorting out the coincidence data packets that indicate an event at the two detectors lying on this line of response.

The sorter 340 provides the image dataset array to an image processing/reconstruction system 342, for example, by way of a communications link 344 to be stored in an image array 346. The image array 346 holds the dataset array for access by an image processor 348 that reconstructs one or more images corresponding to the dataset array. As will be described, images can then be viewed and enhanced using the work station 316. In manner such as described above, other networked workstations may be used as well, which may be remotely located and even include mobile devices and portable workstations.

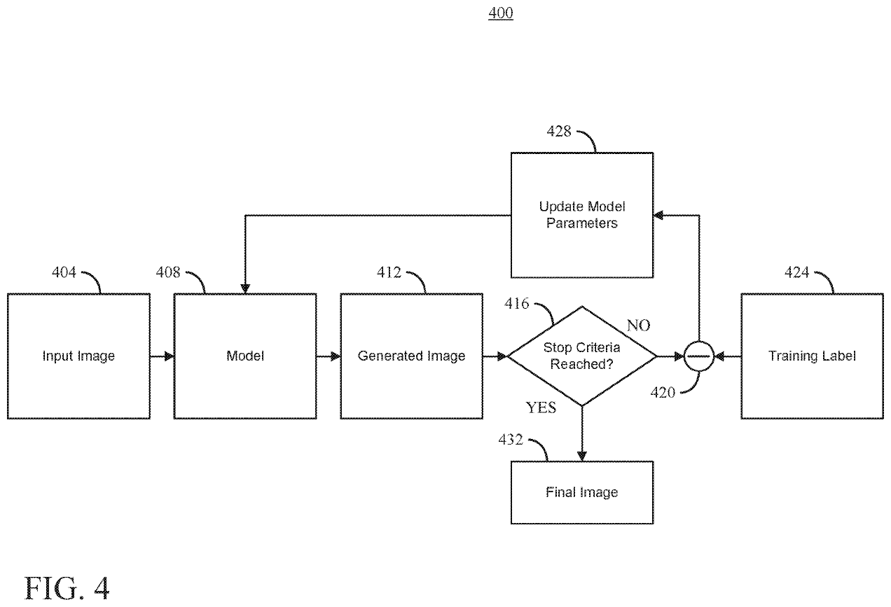

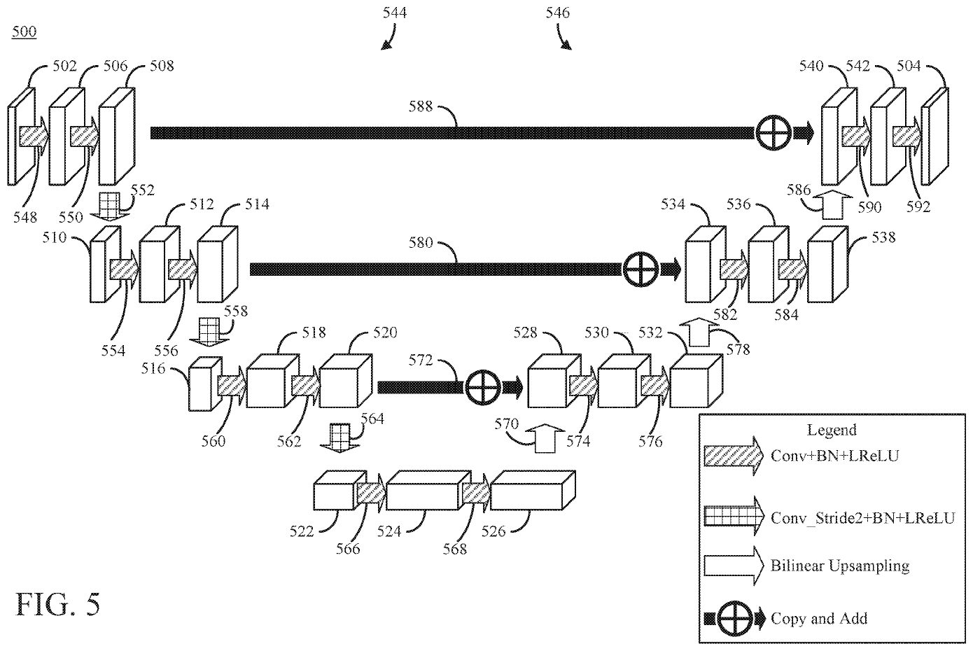

Referring now to FIG. 4, a flow 400 for generating a reconstructed image is shown. The flow 400 can include providing an input image 404 to a model 408. In some configurations, the input image 404 can be a prior image of a patient. The model 408 can generate and output a generated image 412. In some configurations, the model 408 can include a neural network, which will be described in detail below. In some configurations, the model 408 can be a modified 3D U-net as shown in FIG. 5. In some configurations, the generated image 412 can be a reconstructed image (e.g., a reconstructed PET image) and/or a denoised image (e.g., a denoised PET image).

The flow 400 can include determining if stop criteria has been reached 416. In some configurations, the flow 400 can train the model 408 for a predetermined number of epochs before determining that the stop criteria has been reached. For example, the stop criteria can be three hundred epochs. If the stop criteria has been reached (e.g., “NO” at 416), the flow 400 can proceed to loss function calculation 420. In some configurations, the loss function can be an L2 loss function calculated between a training label 424 and the generated image 412. The flow 400 can include calculating a loss function value based on a predetermined loss function that can vary based on the image type of the input image 404 and/or the training label 424, as will be explained below. The flow 400 can include calculating the loss function using the loss function value based on the loss function, the generated image 412, and the training label 424.

The flow can include updating model parameters 428 of the model 412. For example, the flow 400 can adjust weights of the model 408. The updating the model parameters 428 will be described in further detail below. Once the model has been trained using the same input image 404 and the same training label 424 until the stop criteria has been reached (e.g., at 416), the flow 400 can output the last generated image 412 generated by the model 408 as a final image 432. In some configurations, the reconstructed image 432 can be a reconstructed image and/or a denoised image. Specifics of the flow will be discussed below.

In the flow 400, the input image 404 can be prior images of the same patient, rather than random noise used in previous techniques. Instead of calculating the mean squared error (MSE) between the generated image 412 and the training label 424 (which can be considered as the original corrupted image), a training objective function can be formulated based on maximum likelihood estimation derived from imaging physics. As described above, in some configurations, the model 408 can include a modified 3D U-net neural network, which can increase the quality of the final image 432 generated by the flow 400 as compared to other techniques. To stabilize model training, the limited memory Broyden-Fletcher-Goldfarb-Shanno algorithm (L-BFGS algorithm) can be used instead of adaptive stochastic gradient descent (SGD) methods.

In inverse problems, such as image deblurring and image reconstruction, the measured data y∈ [symbol] ^{M} can be assumed as a collection of independent random variables and its mean y̅∈ [symbol] ^{M} is assumed to be related to the original image x∈ [symbol] ^{N} through an affine transform:

y̅=Ax+s (1)

where A∈ [symbol] ^{M×N} is the transformation matrix and s∈ [symbol] ^{M} is a known additive term. Supposing the measured random variable yᵢ follows a distribution of p(yᵢ|x), the log likelihood for the measured data y can be written as:

$$L ( y \vert x ) {{\sum }^{M}}_{i = 1} log p ( {y}_{i} \vert x ) \qquad ( 2 )$$

In this proposed DIPRecon framework, the unknown image x is represented as

x=f(θ|z) (3)

where f represents the neural network, θ are the unknown parameters of the neural network, and z denotes the prior image and is the input to the neural network. When substituting×with the neural network representation in (3), the original data model shown in (1) can be rewritten as

y̅=Af(θ|z)+s (4)

Replacing x by (3), the log likelihood can be expressed using θ as

$$L ( y \vert θ ) = {{\sum }^{M}}_{i = 1} log p ( {y}_{i} \vert f ( θ \vert z ) ) \qquad ( 5 )$$

The maximum likelihood estimate of the unknown image x can be calculated in two steps as

\hat{θ}=arg max L(y|θ) (6)

\hat{x}=f(\hat{θ}|z) (7)

The objective function in (6) is difficult to solve due to the coupling between the transformation matrix and the neural network. The objective function in (6) can be transferred to the constrained format as below

max L(y|x)

s.t.x=f(θ|z) (8)

The augmented Lagrangian format for the constrained optimization problem in (8) can be used as

$${L}_{ρ} = L ( y \vert x ) - \frac{ρ}{2} {‖ x - f ( θ \vert z ) + μ ‖}^{2} + \frac{ρ}{2} {‖ μ ‖}^{2} \qquad ( 9 )$$

which can be solved by the alternating direction method of multipliers (ADMM) algorithm iteratively in three steps:

$${x}^{n + 1} = {argmax}_{x} L ( y \vert x ) - \frac{ρ}{2} {‖ x - f ( {θ}^{n} \vert z ) + {μ}^{n} ‖}^{2} \qquad ( 10 )$$

$${θ}^{n + 1} = {argmin}_{θ} {‖ f ( θ \vert z ) - ( {x}^{n + 1} + {μ}^{n} ) ‖}^{2} \qquad ( 11 )$$

μⁿ⁺¹=μⁿ+xⁿ⁺¹−f(θⁿ⁺¹|z) (12)

### 1) Solving Subproblem (10)

An example of PET image reconstruction is now described as an example application of the flow 400. In PET image reconstruction, A is the detection probability matrix, with Aij denoting the probability of photons originating from voxel j being detected by detector i.s∈ [symbol] ^{M} denotes the expectation of scattered and random events. M is the number of lines of response (LOR). Assuming the measured photon coincidences follow Poisson distribution, the log-likelihood function L(y|x) can be explicitly written as

$$L ( y \vert x ) = {\sum }_{i = 1}^{M} log p ( {y}_{i} \vert x ) = {\sum }_{i = 1}^{M} {y}_{i} log {\overline{y}}_{i} - {\overline{y}}_{i} - log {y}_{i} ! . \qquad ( 13 )$$

Though the measurement data may follow different distributions for other inverse problems, only subproblem (10) needs to be reformulated and the other subproblems (11) and (12) can stay unchanged. Here subproblem (10) is a penalized image reconstruction problem, and the optimization transfer method can be used to solve it. As x in L(y|x) is coupled together, a surrogate function Q_{L}(x|xⁿ) for L(y|x) can be constructed to decouple the image pixels so that each pixel can be optimized independently. Q_{L} (x|xⁿ) can be constructed as follows:

$${Q}_{L} ( x \vert {x}^{n} ) = {\sum }_{j = 1}^{{n}_{j}} {A}_{. j} ( {\hat{x}}_{j , EM}^{n + 1} log {x}_{j} - {x}_{j} ) \qquad ( 14 )$$

where A,j=Σᵢ₌ₗ^{M}Aᵢⱼ and \hat{x}_{j,EM}ⁿ⁺¹ is calculated by

$${\hat{x}}_{j , EM}^{n + 1} = \frac{{x}_{j}^{n}}{{A}_{. j}} {\sum }_{i = 1}^{M} {A}_{ij} \frac{{y}_{i}}{{[ {Ax}^{n} ]}_{i} + {s}_{i}} . \qquad ( 15 )$$

It can be verified that the constructed surrogate function Q_{L}(x|xⁿ) fulfills the following two conditions:

Q_{L}(x|xⁿ)−Q_{L}(xⁿ|xⁿ)≤L(y|x)−L(y|xⁿ) (16)

∇Q_{L}(xⁿ|xⁿ)=∇L(y|xⁿ) (17)

After getting this surrogate function, subproblem (10) can be optimized pixel by pixel. For pixel j, the surrogate objective function for subproblem (10) can be

$$P ( {x}_{j} \vert {x}^{n} ) = {A}_{. j} ( {\hat{x}}_{j , EM}^{n + 1} log {x}_{j} - {x}_{j} ) - {\frac{ρ}{2} [ {x}_{j} - {f ( {θ}^{n} \vert z )}_{j} + {μ}_{j}^{n} ]}^{2} \qquad ( 18 )$$

The final update equation for pixel j after maximizing (18) can be expressed as:

$$\begin{matrix}{x}_{j}^{n + 1} = \frac{1}{2} [ {f ( {θ}^{n} \vert z )}_{j} - {μ}_{j}^{n} - {A}_{. j} / ρ ] \\ + \frac{1}{2} \sqrt{{[ {f ( {θ}^{n} \vert z )}_{j} - {μ}_{j}^{n} - {A}_{. j} / ρ ]}^{2} + 4 {\hat{x}}_{j , EM}^{n + 1} {A}_{. j} / ρ}\end{matrix} \qquad ( 19 )$$

### Solving Subproblem (11)

Subproblem (11) is a nonlinear least square problem. Currently, network training is generally based on first-order methods, such as the Adam algorithm and the Nesterov's accelerated gradient (NAG) algorithm. The L-BFGS algorithm is a quasi-newton method, combining a history of updates to approximate the Hessian matrix. It is not widely used in network training as it requires large batch size to accurately calculate the descent direction, which is less effective than first-order methods for large-scale applications. In this proposed framework, as only the patient's own prior images are employed as the network input, the data size is much smaller than the traditional network training. In our case, the L-BFGS method may be preferred to solve subproblem (11) due to its stability and better performance observed in experiments.

In some configurations, the updating the model parameters 428 can include solving subproblem (10), subproblem (11), and subproblem (12). In some configurations, the loss function calculation 420 can include performing two iterations to solve subproblem (10). The updating the model parameters 428 can include solving subproblem (12) using the solutions to subproblem (10) and subproblem (11). The updating model parameters 428 can include updating the model based on the solution to subproblem (11). Exemplary pseudocode for an algorithm executed at the updating the model parameters 428 to solve subproblem (10) and subproblem (11) is presented Algorithm 1 below.

Algorithm 1

```
Input: Maximum iteration number MaxIt, sub-iteration
  number SubIt1, sub-iteration number Sublt2, network
  initialization θ⁰, Prior image [symbol]
  1: x^{0,Sublt1} = f(θ⁰| [symbol] )
  2: μ⁰ = 0
  3: for n = 1 to MaxIt do
  4: x^{n,0} = x^{n-1,Sublt1}
  5: for m = 1 to SubIt1 do
  6:  $\begin{matrix}{\hat{x}}_{j , EM}^{n , m} = [ {x}_{j}^{n , m - 1} / {A}_{. j} ] {\sum }_{i = 1}^{M} {A}_{ij} \frac{{y}_{i}}{{[ {Ax}^{n , m - 1} ]}_{i} + {s}_{i}} , \\ where {A}_{. j} = {\sum }_{i = 1}^{M} {A}_{ij}\end{matrix}$
  7:  $\begin{matrix}{x}_{j}^{n , m} = \frac{1}{2} [ {f ( {θ}^{n - 1} \vert z )}_{j} - {μ}_{j}^{n - 1} - {A}_{. j} / ρ ] + \\ \frac{1}{2} \sqrt{( {[ {f ( {θ}^{n - 1} \vert z )}_{j} - {μ}_{j}^{n} - {A}_{. j} / ρ ]}^{2} + 4 {\hat{x}}_{j , EM}^{n , m} {A}_{. j} / ρ )}\end{matrix}$
  8: end for
  9: x_{label}ⁿ = x^{n,SubIt1} + μⁿ⁻¹
10: Running L-BFGS algorithm SubIt2 iterations to train
    the network, get θⁿ = arg min_{θ} || f(θ| [symbol] ) − x_{label}ⁿ||²
11: μⁿ = μⁿ⁻¹ + x^{n,SubIt1} − f(θⁿ | [symbol] )
12: end for
13: return \hat{x} = f(θ^{MaxIt}| [symbol] )
```

Referring now to FIG. 5, an exemplary neural network 500 for use in a deep learning framework is shown. In some configurations, the model 408 in FIG. 4 can include the neural network 500. In some configurations, the neural network 500 can be trained to generate reconstructed PET images. PET is a molecular imaging modality widely used in neurology studies. The image resolution of current PET scanners is still limited by various physical degradation factors. Improving PET image resolution is essential for many applications, such as dopamine neurotransmitter imaging, brain tumor staging and early diagnosis of Alzheimer's disease. For the past decades, various efforts are focusing on using MR or CT to improve PET image quality. In this work, the anatomically-aided PET image reconstruction problem was presented as an example to demonstrate the effectiveness of the proposed DIPRecon framework. Compared with the state-of-the-art kernel-based method and the penalized reconstruction based on a neural network penalty, DIPRecon shows superior performance both visually and quantitatively in simulation and real data experiments.

In some configurations, the neural network 500 can include certain components of the 3D U-Net with a number of different components. In some configurations, the neural network 500 can receive an input image 502. In some configurations, the input image 502 can be a single channel three dimensional image (e.g., an MR image of a patient). In some configurations, the neural network 500 can generate and output a predicted image 504 (e.g., a reconstructed PET image). In some configurations, the predicted image 504 can be a single channel three dimensional image. As will be described below, for certain imaging types (e.g., DECT images), the predicted image 504 can be a multi-channel three dimensional image (e.g., a two channel image). In some configurations, the neural network 500 can include a number of feature maps coupled together by a combination of different layers. In some configurations, the neural network 500 can include a first feature map 506, a second feature map 508, a third feature map 510, a fourth feature map 512, a fifth feature map 514, a sixth feature map 516, a seventh feature map 518, an eighth feature map 520, a ninth feature map 522, a tenth feature map 524, a eleventh feature map 526, a twelfth feature map 528, a thirteenth feature map 530, a fourteenth feature map 532, a fifteenth feature map 534, a sixteenth feature map 536, a seventeenth feature map 538, an eighteenth feature map 540, and a nineteenth feature map 542.

As described above, the feature maps 506-542 can be coupled together using a number of different layers. In some configurations, the layers can include a convolutional layer (e.g., a 3×3×3 3D convolutional layer), a batch normalization (BN) layer, a leaky rectified linear unit (LReLU) layer, a convolutional layer with a stride value greater than one (e.g., a 3×3×3 3D convolutional layer with stride 2×2×2), a bilinear interpolation layer (e.g. a 2×2×2 bilinear interpolation layer), and an identity mapping layer. Each 3×3×3 3D convolutional layer with stride 2×2×2 can be used to down-sample certain feature maps, and the 2×2×2 bilinear interpolation layer can be used to up-sampling certain feature maps. Each identity mapping layer can map features in a left-side encoder path 544 of the neural network 500 to features in a right-side decoder path 546 of the neural network 500. In some configurations, the identity mapping layer can include skip connections and can add features on the left-side encoder path 544 to features in the right-side decoder path 546.

Certain layers can provide advantages over a standard 3D U-Net. In particular, convolutional layers with stride two to down-sample the input image 502 instead of using max pooling in order to construct a fully convolutional neural network. A fully convolutional network may be trained more easily than if max-pooling layers were used in place of the convolutional layers with stride two. Additionally, the fully convolutional layer may provide better image generation results (e.g., better contrast, more accurate reconstructions, etc.). The identity mapping layers with skip connections to link the encoder path 544 and the decoder path 546 instead of concatenating can reduce the number of training parameters, which can improve the training speed of the neural network 500. The bi-linear interpolation layer can be used instead of deconvolution upsampling in order to reduce potential checkboard artifact. Additionally, leaky ReLU layers can be used instead of ReLU layers. As compared to standard ReLU layers, leaky ReLU layers can may provide better information flow, and as a result of improved information flow, better training.

The layers may be arranged into groups of common layer arrangements. For example, a layer group can include a convolutional layer (e.g., a convolutional layer with a stride length of 1), a BN layer, and an LReLU layer. In some configurations, the neural network 500 can include a first layer group 548, a second layer group 550, a third layer group 552, a fourth layer group 554, a fifth layer group 556, a sixth layer group 558, a seventh layer group 560, an eighth layer group 562, a ninth layer group 564, a tenth layer group 566, a eleventh layer group 568, a twelfth layer group 570, a thirteenth layer group 572, a fourteenth layer group 574, a fifteenth layer group 576, a sixteenth layer group 578, a seventeenth layer group 580, an eighteenth layer group 582, a nineteenth layer group 584, a twentieth layer group 586, a twenty-first layer group 588, a twenty-second layer group 590, and a twenty-third layer group 592.

In some configurations, the input image 502 can be coupled to the first feature map 506 by the first layer group 548 including a convolutional layer (e.g., a convolutional layer with a stride length of 1), a BN layer, and an LReLU layer. In some configurations, the first feature map 506 can be coupled to the second feature map 508 by the second layer group 550 including a convolutional layer (e.g., a convolutional layer with a stride length of 1), a BN layer, and an LReLU layer. In some configurations, the first feature map 506 and the second feature map 508 can include sixteen channels. In some configurations, the second feature map 508 can be downsampled to the third feature map 510 by the third layer group 552 including a convolutional layer with stride 2×2×2, a BN layer, and an LReLU layer.

In some configurations, the third feature map 510 can be coupled to the fourth feature map 512 by the fourth layer group 554 including a convolutional layer (e.g., a convolutional layer with a stride length of 1), a BN layer, and an LReLU layer. In some configurations, the fourth feature map 512 can be coupled to the fifth feature map 514 by the fifth layer group 556 including a convolutional layer (e.g., a convolutional layer with a stride length of 1), a BN layer, and an LReLU layer. In some configurations, the fourth feature map 512 and the fifth feature map 514 can include thirty-two channels. In some configurations, the fifth feature map 514 can be downsampled to the sixth feature map 516 by the sixth layer group 558 including a convolutional layer with stride 2×2×2, a BN layer, and an LReLU layer.

In some configurations, the sixth feature map 516 can be coupled to the seventh feature map 518 by the seventh layer group 560 including a convolutional layer (e.g., a convolutional layer with a stride length of 1), a BN layer, and an LReLU layer. In some configurations, the seventh feature map 518 can be coupled to the eighth feature map 520 by the eighth layer group 562 including a convolutional layer (e.g., a convolutional layer with a stride length of 1), a BN layer, and an LReLU layer. In some configurations, the seventh feature map 518 and the eighth feature map 520 can include sixty-four channels. In some configurations, the eighth feature map 520 can be downsampled to the ninth feature map 522 by the ninth layer group 564 including a convolutional layer with stride 2×2×2, a BN layer, and an LReLU layer.

In some configurations, the ninth feature map 522 can be coupled to the tenth feature map 524 by the tenth layer group 566 including a convolutional layer (e.g., a convolutional layer with a stride length of 1), a BN layer, and an LReLU layer. In some configurations, the tenth feature map 524 can be coupled to the eleventh feature map 526 by the eleventh layer group 568 including a convolutional layer (e.g., a convolutional layer with a stride length of 1), a BN layer, and an LReLU layer. In some configurations, the tenth feature map 524 and the eleventh feature map 526 can include one hundred and twenty-eight channels. In some configurations, the eleventh feature map 526 can be upsampled to the twelfth feature map 528 by the twelfth layer group 570 including a bilinear interpolation layer (e.g. a 2×2×2 bilinear interpolation layer).

In addition to upsampling the eleventh feature map 526, the twelfth feature map 528 can be coupled to the eighth feature map 520 by the thirteenth layer group 572 including skip connections (e.g., copy and add skip connections) to receive at least some values (e.g., features that are not skipped) from the eighth feature map 520. The values from the eighth feature map 520 can be added to the upsampled values of the eleventh feature map 526 to generate the twelfth feature map 528.

In some configurations, the twelfth feature map 528 can be coupled to the thirteenth feature map 530 by the fourteenth layer group 574 including a convolutional layer (e.g., a convolutional layer with a stride length of 1), a BN layer, and an LReLU layer. In some configurations, the thirteenth feature map 530 can be coupled to the fourteenth feature map 532 by the fifteenth layer group 576 including a convolutional layer (e.g., a convolutional layer with a stride length of 1), a BN layer, and an LReLU layer. In some configurations, the twelfth feature map 528 and the thirteenth feature map 530 can include sixty-four channels. In some configurations, the fourteenth feature map 532 can be upsampled to the fifteenth feature map 534 by the sixteenth layer group 578 including a bilinear interpolation layer (e.g. a 2×2×2 bilinear interpolation layer).

In addition to upsampling the fourteenth feature map 532, the fifteenth feature map 534 can be coupled to the fifth feature map 514 by the seventeenth layer group 580 including skip connections (e.g., copy and add skip connections) to receive at least some values (e.g., features that are not skipped) from the fifth feature map 514. The values from the fifth feature map 514 can be added to the upsampled values of the fourteenth feature map 532 to generate the fifteenth feature map 534.

In some configurations, the fifteenth feature map 534 can be coupled to the sixteenth feature map 536 by the eighteenth layer group 582 including a convolutional layer (e.g., a convolutional layer with a stride length of 1), a BN layer, and an LReLU layer. In some configurations, the sixteenth feature map 536 can be coupled to the seventeenth feature map 538 by the nineteenth layer group 584 including a convolutional layer (e.g., a convolutional layer with a stride length of 1), a BN layer, and an LReLU layer. In some configurations, the fifteenth feature map 534 and the sixteenth feature map 536 can include thirty-two channels. In some configurations, the seventeenth feature map 538 can be upsampled to the eighteenth feature map 540 by the twentieth layer group 586 including a bilinear interpolation layer (e.g. a 2×2×2 bilinear interpolation layer).

In addition to upsampling the seventeenth feature map 538, the eighteenth feature map 540 can be coupled to the second feature map 508 by the twenty-first layer group 588 including skip connections (e.g., copy and add skip connections) to receive at least some values (e.g., features that are not skipped) from the second feature map 508. The values from the second feature map 508 can be added to the upsampled values of the seventeenth feature map 538 to generate the eighteenth feature map 540.

In some configurations, the eighteenth feature map 540 can be coupled to the nineteenth feature map 542 by the twenty-second layer group 590 including a convolutional layer (e.g., a convolutional layer with a stride length of 1), a BN layer, and an LReLU layer. In some configurations, the nineteenth feature map 542 can be coupled to the predicted image 504 by the twenty-third layer group 592 including a convolutional layer (e.g., a convolutional layer with a stride length of 1), a BN layer, and an LReLU layer. In some configurations, the eighteenth feature map 540 and the nineteenth feature map 542 can include sixteen channels. In some configurations, the input image 502 and the predicted image 504 can have the same resolution (e.g., 192×192×128). The resolution of the input image 502 can vary based on the type of imaging device used to generate the input image 502. Through experimentation it was found that using 3D convolution for 3D image reconstruction performed better than using 2D convolution with multiple axial slices in the input channels. To enable a non-negative constraint on the reconstructed image, an ReLU layer (e.g., LReLU) can be added before the predicted image 504. In addition, it was found that compared to the network without encoder and decoder path, the U-net structure could save more GPU memory because the spatial size is reduced due to the encoder path.

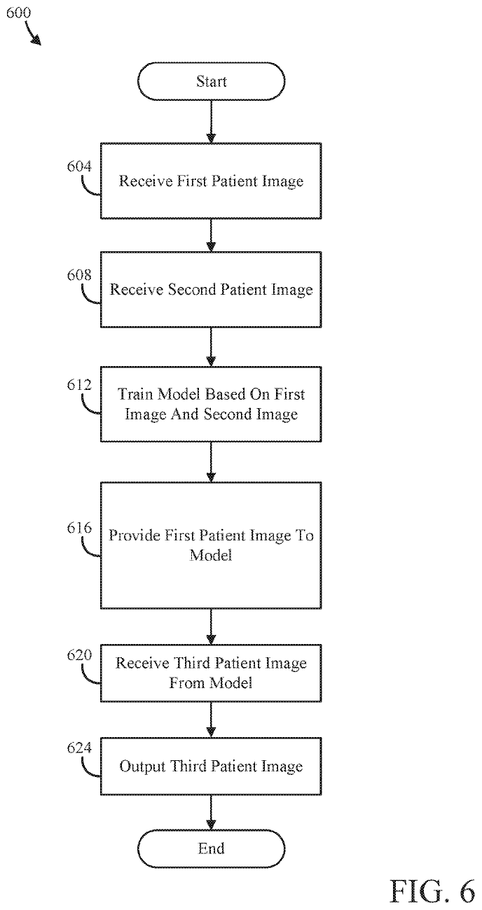

Referring now to FIG. 6, an exemplary process 600 for training a model is shown. In some configurations, the model can include the neural network 500 in FIG. 5. In some configurations, the process 600 can train the model to generate a reconstructed PET image using a MR image and a raw PET image (e.g., a raw sinogram) without any other training data. In some configurations, the medical image analysis application 132 can include the process 600. In some configurations, the processes 600 can be implemented as computer readable instructions on a memory or other storage medium and executed by a processor. In some configurations, the model can be a neural network without any pretraining. For example, the model can include randomized values for all trainable parameters.

At 604, the process 600 can receive a first patient image. In some configurations, the first patient image can be an MR image associated with the patient. In some configurations, the first patient image can be the input image 404 in FIG. 4. In some configurations, the first patient image can be a single channel image having pixel intensities normalized to a range of [0, 1].

At 608, the process 600 can receive a second patient image. In some configurations, the second patient image can be a raw PET image associated with the patient. In some configurations, the second patient image can be previously acquired concurrently and/or simultaneously along with the first patient image.

At 612, the process 600 can train the model based on the first patient image and the second patient image. In some configurations, the first patient image and the second patient image are formed from image data acquired from different image modalities, such as MRI, CT, ultrasound, or PET. As described above, in some configurations, the model can include the neural network 500 in FIG. 5. In some configurations, the process 600 can provide the first patient image to the model (e.g., as the input image 502), receive an output image from the model (e.g., the predicted image 504). Based on the output image and the second patient image, the process 600 can update weights of parameters in the model. In some configurations, the process 600 can execute Algorithm 1 (as described above) at 612. In some configurations, in algorithm 1, the process 600 can set variable MaxIt to three hundred, variable SubIt1 to two, and variable SubIt2 to ten.

In some configurations, the process 600 can pretrain the model for a predetermined number of epochs (e.g., three hundred epochs), but pretraining is not required in order to train the model to sufficiently generate reconstructed PET images and/or other images.

In some configurations, for a certain number of iterations (e.g., MaxIt and/or three hundred), the process 600 can provide the first patient image to the model, receive an output image from the model, and update the model based on the output image and the second patient image. The process 600 can solve objection function (6) by breaking equation (9) into the three subproblems (10)-(12) using ADMM as described above. The process 600 can iteratively solve both subproblems (10)-(12) for a predetermined number of iterations (e.g., MaxIt and/or three hundred) in order to train the network based on the first patient image (e.g., z) and the second patient image (e.g., y). The process 600 can iteratively solve equations (15) and (19) for a predetermined number of iterations (e.g., SubIt1 and/or two) to solve subproblem (10). It is noted that subproblem (10) is solved in part based on the assumption that the photon coincidences in the raw PET image follow the Poisson distribution. Penalty parameter p in equations (15) and (19) can be predetermined (e.g., 3×10⁻³). The process 600 can iteratively solve subproblem (11) for a predetermined number of iterations (e.g., SubIt2 and/or ten) using the L-BFGS algorithm and the resulting x^{n,SubIt1} from the last iteration of equation (19). The process 600 can update the model based on the θⁿ obtained when solving equation (19).

At 616, the process 600 can provide the first patient image to the model. At 616, the model is trained and can be referred to as a trained model.

At 620, the process 600 can receive a third patient image from the model. In some configurations, the third patient image can be a reconstructed PET image that the model generates based on the first patient image (e.g., an MR image).

At 624, the process 600 can output the third patient image to at least one of a memory or a display. In some configurations, at 624, the process 600 can cause the third patient image to be displayed on a display (e.g., the display 108). In some configurations, at 624, the process 600 can generate a report based on the third patient image. In some configurations, at 624, the process 600 can output the report to at least one of a memory or a display. In some configurations, at 624, the process 600 can cause the report to be displayed on a display (e.g., the display 108).

It is understood that at least some of the steps included in the process 1600 can be rearranged and/or executed in parallel.

### Testing and Experimentation

A number of experiments to determine values of certain operating parameters (e.g., penalty parameter, number of epochs, etc.) are now described. To stabilize the network training, before training, the flow 400 and/or the process 600 can include scaling the intensity of a PET image to the range [0, 1]. Due to the non-convexity of neural network training, it is generally better to assign good initials to the network parameters before being trained inside the iterative reconstruction loop. Comparisons between the results with and without pre-training are shown in the supplemental materials. In some configurations, the neural network 500 can be trained by running ML EM algorithm for 60 iterations, and then used it as x_{label} to train the network based on

$$\hat{θ} = arg {min}_{θ} {\sum }_{i} ‖ {x}_{label}^{i} - f ( θ \vert {z}^{i} ) ‖ \qquad ( 20 )$$

Using MR images as network input, where f: [symbol] → [symbol] represents the neural network, θ∈ [symbol] ^{L} indicates the trainable variables, zⁱ∈ [symbol] ^{N} is the network input for the ith training pair, and x_{label}ⁱ∈ [symbol] denotes the ith training label. For CNN, θ contains the convolutional filters and the bias items from all layers.

Network pretraining was run for three hundred epochs using the L-BFGS algorithm based on minimizing (20). As the penalty parameter has a large impact on the convergence speed, the convergence of the log-likelihood L(y|f(\hat{θ}|z)) was examined to determine the penalty parameter used in practice. A log-likelihood curve was constructed for different penalty parameter ρ values, and based on the convergence speed and stability of the likelihood, ρ=3×10⁻³ was chosen.

The neural network 500 trained using the process 600 was compared with the Gaussian postfiltering method, the penalized reconstruction method based on including a pre-trained neural network in the penalty, and the kernel method. One technique of incorporating the trained network into iterative reconstruction framework is include it in the penalty item. The objective function is:

$$\hat{x} = {argmax}_{x} L ( y \vert x ) - β {‖ x - f ( {θ}_{0} \vert z ) ‖}^{2} \qquad ( 21 )$$

Here f(θ₀|z) is the pre-trained network using noisy PET images (e.g., raw PET images) as labels and the pre-trained network parameters θ₀ is fixed during the reconstruction process. This method is denoted as CNNPenalty method. Interestingly, the objective function shown in (21) becomes the same as subproblem (13) by replacing f(θ₀|z) with f(θⁿ|z)+μⁿ. In the flow 400 and the process 600, one difference between the CNNPenalty method and the process 600 is that μ was fixed to 0 and the network updating step (e.g., equation (11)) was skipped. By this setting, the effects of updating the network parameters inside the reconstruction loop can be understood. For the kernel method, the (i,j)th element of the kernel matrix K_{z} is

$${k}_{ij} = exp ( - \frac{{‖ {f}_{i} - {f}_{j} ‖}^{2}}{2 {N}_{f} {σ}^{2}} ) \qquad ( 22 )$$

where fᵢ∈ [symbol] ^{N}^{_{f}} and fⱼ∈ [symbol] ^{N}^{_{f}} represents the feature vectors of voxel i and voxel j from the MR prior image z, σ² is the variance of the prior image, and N_{f} is the number of voxels in a feature vector. For efficient computation, the kernel matrix was constructed using a K-Nearest-Neighbor (KNN) search in a 7×7×7 search window with K=50. A 3×3×3 local patch was extracted for each voxel to form the feature vector. During image reconstruction, the linear coefficients θ were computed using iterative update as

$${θ}^{n + 1} = \frac{{θ}^{n}}{{( {AK}_{z} )}^{T} {1}_{M}} [ {( {AK}_{z} )}^{T} \frac{y}{( {AK}_{z} ) {θ}^{n} + s} ] \qquad ( 23 )$$

and the final output image is \hat{x}=K_{z}\hat{θ}. The kernel method is denoted as KMRI in later comparisons.

In testing, the network training was done in GPU (NVIDIA GTX 1080 Ti) and the image reconstruction was implemented in CPU with 16 threads (Intel Xeon E5-2630 v3). The neural network was implemented using TensorFlow 1.4. The L-BFGS algorithm was implemented using the TensorFlow “ScipyOptimizerinterface” to call “L-BFGS-B” method from scipy library running in CPU mode. Ten previous iterations were used in L-BFGS to approximate the Hessian matrix.

### Brain Phantom Simulation

A 3D brain phantom from BrainWeb was used in the simulation. Corresponding T1-weighted MR image was used as the prior image. The voxel size was 2×2×2 mm³ and the phantom image size was 128×128×105. The input to the network was cropped to 128×128×96 to reduce GPU memory usage. To simulate mismatches between the MR and PET images, twelve hot spheres of diameter 16 mm were inserted into the PET image as tumor regions, which are not visible in the MR image. The time activity curves of blood, gray matter, white matter and tumor were simulated based on a two-tissue compartment model. The computer simulation modeled the geometry of a Siemens mCT scanner, and the system matrix was generated using the multi-ray tracing method. In this experiment, the last five minute frame of a one-hour FDG scan was used as the ground-truth image. Noise-free sinogram data were generated by forward-projecting the ground-truth image using the system matrix and the attenuation map. Poisson noise was then introduced to the noise-free data by setting the total count level to be equivalent to last 5 min scan with 5 mCi injection. Uniform random events were simulated and accounted for 30 percent of the noise-free data. Scatters were not included. For quantitative comparison, contrast recovery coefficient (CRC) vs. the standard deviation (STD) curves were plotted based on reconstructions of twenty independent and identically distributed (i.i.d) realizations. The CRC was computed between selected gray matter regions and white matter regions as

$$CRC = \frac{1}{R} {\sum }_{r = 1}^{R} ( \frac{{\overline{a}}_{r}}{{\overline{b}}_{r}} - 1 ) / ( \frac{{a}^{true}}{{b}^{true}} - 1 ) \qquad ( 24 )$$

Here R is the number of realizations and is set to 20, āᵣ=1/KₐΣₖ₌₁^{K}^{ₐ} a_{r,k} is is the average uptake of the gray matter over Kₐ ROIs in realization r. The ROIs were drawn in both matched gray matter regions and the tumor regions. For the case of matched gray matter, Kₐ=10. For the tumor regions, Kₐ=12. When choosing the matched gray matter regions, only those pixels inside the predefined 20-mm-diameter spheres and containing 80% of gray matter were included. b̅ᵣ=1/K_{b}Σₖ₌₁^{K}^{_{b}}b_{r,k} is the average value of the background ROIs in realization r, and K_{b}=37 is the total number of background ROIs. The background ROIs were drawn in the white matter. The background STD was computed as

$$STD = \frac{1}{{K}_{b}} {\sum }_{k = 1}^{{K}_{b}} \frac{\sqrt{\frac{1}{R - 1} {\sum }_{r = 1}^{R} {( {b}_{r , k} - {\overline{b}}_{k} )}^{2}}}{{\overline{b}}_{k}} \qquad ( 25 )$$

where b̅ₖ=1/R Σᵣ₌₁^{R}b_{r,k} is the average of the background ROI means over realizations.

The network structure for the simulated data set is the same as the network structure shown in FIG. 5, with input and output size set to 128×128×96.

### Real Brain Data Sets

In this experiment, a 70-minutes dynamic PET scan of a human subject acquired on a Siemens Brain MR-PET scanner after 5 mCi FDG injection was employed in the real data evaluation. The data were reconstructed with an image array of 256×256×153 and a voxel size of 1.25×1.25×1.25 mm³. The input to the network was cropped to 192×192×128 to reduce GPU memory usage. A simultaneous acquired T1-weighted MR image having the same image array and voxel size as the PET image was used as the prior image. Correction factors for random, scatters were estimated using the standard software provided by the manufacturer and included during reconstruction. The motion correction was performed in the line-of-response (LOR) space based on the simultaneously acquired MR navigator signal. Attenuation was derived from T1-weighted MR image using the SPM based atlas method. To generate multiple realizations for quantitative analysis, the last 40 minutes PET data were binned together and resampled with a ⅛ ratio to obtain 20 i.i.d. datasets that mimic 5-minutes frames. As the ground truth of the regional uptake is unknown, a hot sphere with diameter 12.5 mm, mimicking a tumor, was added to the PET data (invisible in the MRI image). This addition was intended to simulate the case where MRI and PET information does not match. The TAC of the hot sphere was set to the TAC of the gray matter, so the final TAC of the simulated tumor region is higher than that of the gray matter because of the superposition. The simulated tumor image of the last 40 minutes was forward-projected to generate a set of noise free sinograms, including detector normalization and patient attenuation. Randoms and scatters from the inserted tumor were not simulated as they would be negligible compared with the scattered and random events from the patient background. Poisson noise was then introduced and finally the tumor sinograms were added to the original patient sinograms to generate the hybrid real data sets. For tumor quantification, images with and without the inserted tumor were reconstructed and the difference was taken to obtain the tumor only image and compared with the ground truth. The tumor contrast recovery (CR) was calculated as

$$CR = \frac{1}{R} {\sum }_{r = 1}^{R} {\overline{l}}_{r} / {l}_{true} \qquad ( 26 )$$

where l̅ᵣ is the mean tumor uptake inside the tumor ROI, lₜᵣᵤₑ is the ground truth of the tumor uptake, and R is the number of the realizations. For the background, eleven circular ROIs with a diameter of 12.5 mm were drawn in the white matter and the standard deviation was calculated according to (25). The network structure for the real data set was the model 500 in FIG. 5.

### Results

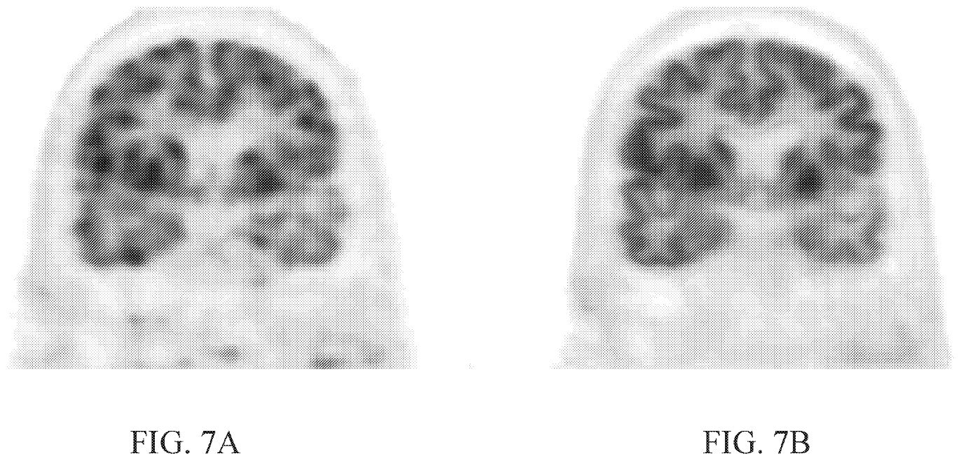

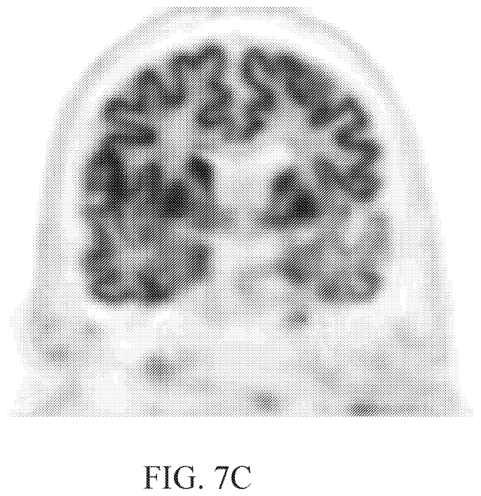

To test the effectiveness of the conditional DIP framework, an experiment was performed by using either the uniform random noise or the patient's MR prior image as the network input. ML EM reconstruction of the real brain data at 60th iteration was treated as the label image. 300 epochs were run for network training using the L-BFGS algorithm. Referring now to FIG. 7A, a patient image generated using the DIP framework with random noise as network input is illustrated. FIG. 7B illustrates a patient image generated using the conditional DIP framework with an MR image of the patient as network input. FIG. 7C illustrates a patient image generated using the neural network 500 trained using the process 600 and using the MR image of the patient as network input.

When the input is random noise (e.g., in FIG. 7A), the image is smooth, but some cortex structures cannot be recovered. When the input is MR image (e.g., FIGS. 7B and 7C), more cortex structures show up. This comparison demonstrates the benefits of including the patient's prior image as the network input. FIG. 7C shows the benefits of the process 600 to train the neural network. Using the process 600 can include the neural network 500 into reconstruction framework as which can allow the trained neural network 500 to recover more cortex details and generate higher contrast between the white matter and gray matter as compared to the conditional DIP framework.

The behaviors of different optimization algorithms under the conditional DIP framework were also compared. The Adam, NAG, and L-BFGS algorithms were compared regarding the pre-training process using the real brain data. When comparing different algorithms, the normalized cost value was computed, which is defined as

$${L}_{n} = \frac{{ϕ}_{Adam}^{ref} - {ϕ}^{n}}{{ϕ}_{Adam}^{ref} - {ϕ}_{Adam}^{1}} \qquad ( 27 )$$

where ϕ_{Adam}^{ref} and ϕ_{Adam}¹ is the cost value defined in (20) after running Adam for seven hundred iterations and one iteration, respectively.

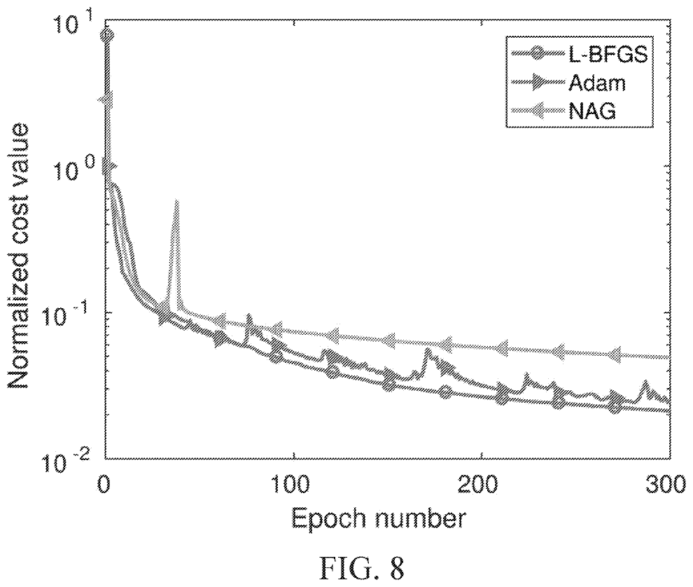

Referring now to FIG. 8, a plot of the normalized cost value curves for different algorithms is illustrated. The L-BFGS algorithm is monotonic decreasing while the Adam algorithm is not due to the adaptive learning rate implemented. The NAG algorithm is slower than the other two algorithms. The reason why L-BFGS is faster is due to its using the approximated Hessian matrix, which makes it closer to a second-order optimization algorithm, while both the Adam and NAG methods are first-order methods. The monotonic property is another advantage of L-BFGS algorithm. Due to the monotonic property, the network output using the L-BFGS algorithm is more stable and less influenced by the image noise when running multiple realizations. Faster convergence speed and better quantitative results are the reasons the L-BFGS algorithm can solve subproblem (11) and perform the initial network training.

### Simulation Results

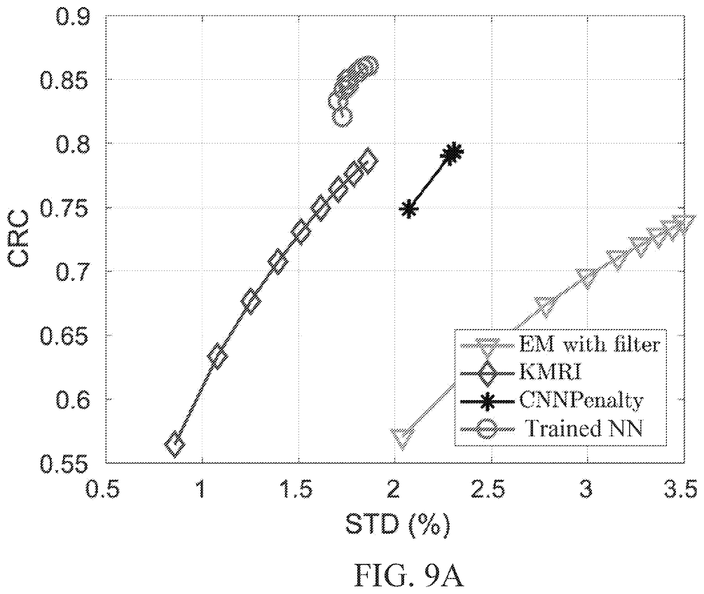

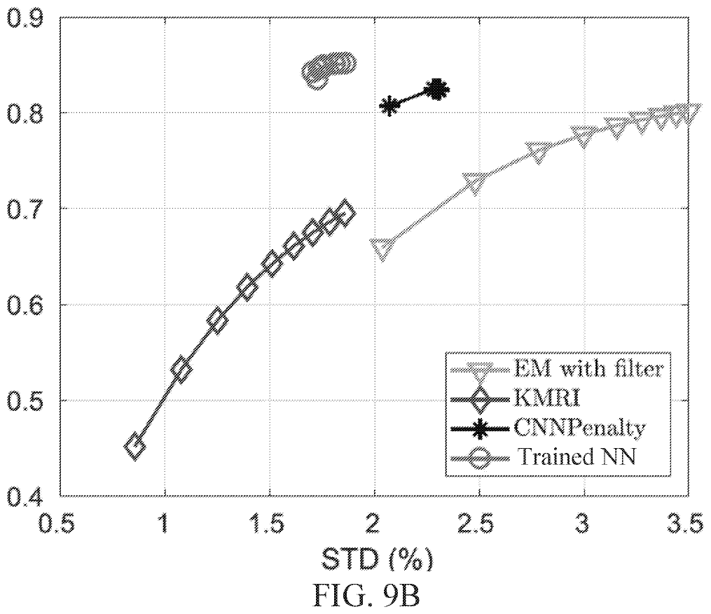

A simulation was performed to reconstruct PET images for three MR images having different orthogonal views. The techniques compared were the EM+filter method, the kernel method, the CNNPenalty method, and the technique of training the neural network 500 using the process 600. The kernel method and the neural network 500 trained using the process 600 both generated images with more cortex structures and have lower noise compared to the EM-plus-filter method and the CNNPenalty method. Compared with the kernel method, the neural network 500 trained using the process 600 recovered even more details of the cortices, and the white matter regions were cleaner. Furthermore, compared to the kernel method, the tumor uptake using the neural network 500 trained using the process 600 is higher and the tumor shape is closer to the ground truth. In this simulation set-up, there are no tumor signals in the prior MRI image, and the neural network 500 trained using the process 600 can still recover PET signals, which is a sign of robustness to potential mismatches between PET and prior images. Besides, by comparing the CNNPenalty method and the neural network 500 trained using the process 600, updating the network parameters inside the reconstruction loop can recover more brain structures and reduce the image noise than other reconstruction methods. Referring now to FIGS. 9A and 9B, plots of CRC-STD curves for different methods are shown. FIG. 9A illustrates plotted CRC-STD curves at the gray matter region in the simulation dataset. FIG. 9B illustrates plotted CRC-STD curves at the gray matter region in the simulation dataset. Markers are plotted every ten iterations. The lowest CRC point corresponds to the 20th iteration for each methods. For both the gray matter region and the tumor region, at fixed STD, the CRC of the neural network 500 trained using the process 600 is higher than other methods. This observation quantitatively shows that the neural network 500 trained using the process 600 (denoted as “Trained NN”) out-performs other methods.

### Real Data Results

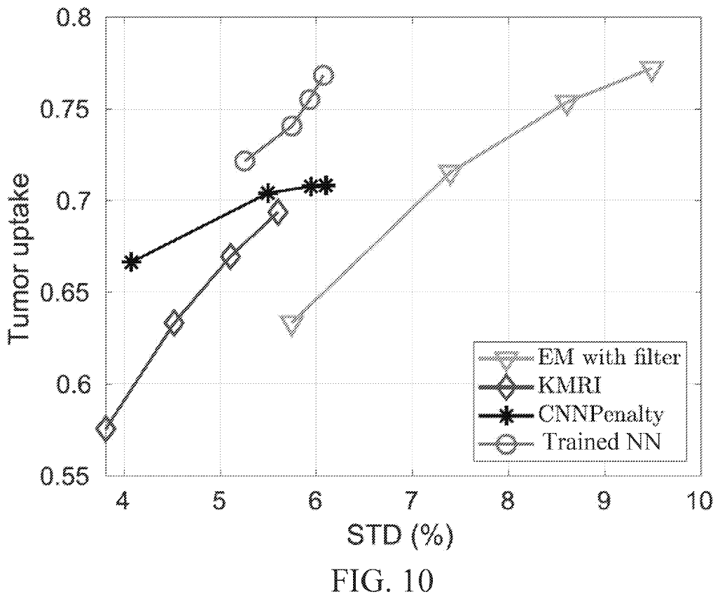

For the real brain dataset, high-count images were reconstructed from the combined 40-min scanning for reference. Compared to the EM-plus-filter method and the CNNPenalty method, the kernel method and the neural network 500 trained using the process 600 can recover more cortex details. Additionally, image noise in the white matter is reduced. The cortex shape using the neural network 500 trained using the process 600 is clearer than the kernel method. For the tumor region which is unobserved in the MR image, the uptake is higher in the neural network 500 trained using the process 600 as compared with the kernel method. FIG. 10 shows the CR-STD curves for different methods. Clearly, the neural network 500 trained using the process 600 (denoted as “Trained NN”) has the best CR-STD trade-off compared with the other methods.

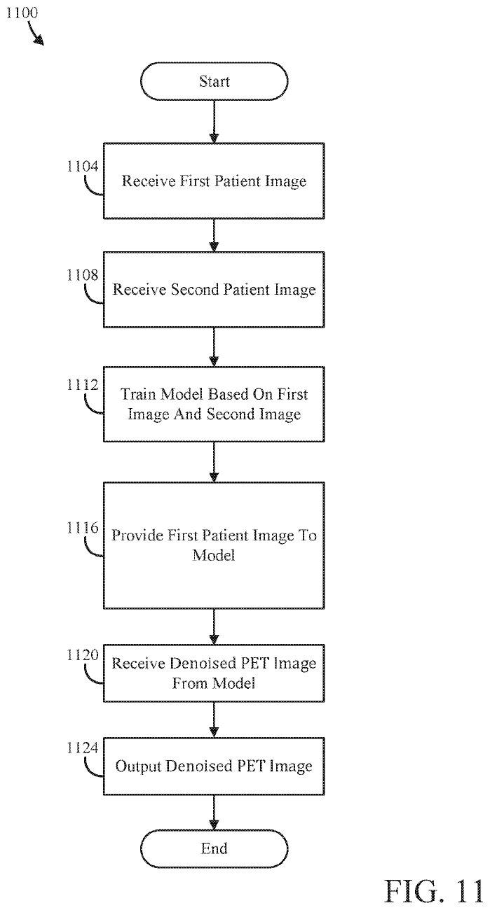

Referring now to FIG. 11, another exemplary process 1100 for training a model is shown. In some configurations, the model can include the neural network 500 in FIG. 5. In some configurations, the process 1100 can train the model to generate a reconstructed PET image using an MR image and/or a CT image, along with a noisy PET image. In some configurations, the medical image analysis application 132 can include the process 1100. In some configurations, the processes 1100 can be implemented as computer readable instructions on a memory or other storage medium and executed by a processor. In some configurations, the model can be a neural network without any pretraining. For example, the model can include randomized values for all trainable parameters.

At 1104, the process 1100 can receive a first patient image. In some configurations, the first patient image can be an MR image associated with the patient. In some configurations, the first patient image can be a T1-weighted MR image. In some configurations, the first patient image can be a CT image associated with the patient. In some configurations, the first patient image can be the input image 404 in FIG. 4. In some configurations, the first patient image can be a single channel image having pixel intensities normalized to a range of [0, 1].

At 1108, the process 1100 can receive a second patient image. In some configurations, the second patient image can be a noisy PET image associated with the patient. In some configurations, the noisy PET image can be previously generated using the maximum likelihood expectation maximization (MLEM) algorithm running a predetermined number of iterations (e.g., forty iterations). In some configurations, the second patient image can be previously acquired concurrently and/or simultaneously along with the first patient image.

At 1112, the process 1100 can train the model based on the first patient image and the second patient image. Again, in some configurations, the first patient image and the second patient image may be images produced by differing imaging modalities. As described above, in some configurations, the model can include the neural network 500 in FIG. 5. In some configurations, the process 1100 can provide the first patient image to the model (e.g., as the input image 502), receive an output image from the model (e.g., the predicted image 504). Based on the output image and the second patient image, the process 1100 can update weights of parameters in the model. In some configurations, the process 1100 can train the model by optimizing equation (5). Equation (5) can be formulated and solved based on the assumption that the photon coincidences in the noisy PET image follow a Gaussian distribution.

At 1116, the process 1100 can provide the first patient image to the model. At 1116, the model is trained and can be referred to as a trained model.

At 1120, the process 1100 can receive a denoised PET image from the model. In some configurations, the model can generate the denoised PET image based on the first patient image (e.g., an MR image).

At 1124, the process 1100 can output the denoised PET image to at least one of a memory or a display. In some configurations, at 1124, the process 1100 can cause the denoised PET image to be displayed on a display (e.g., the display 108). In some configurations, at 1124, the process 1100 can generate a report based on the denoised PET image. In some configurations, at 1124, the process 1100 can output the report to at least one of a memory or a display. In some configurations, at 1124, the process 1100 can cause the report to be displayed on a display (e.g., the display 108).

It is understood that at least some of the steps included in the process 1100 can be rearranged and/or executed in parallel.

In testing, denoised PET images generated in accordance with the process 1100 were compared to a number of other denoising techniques. In a simulation study, contrast recovery coefficient (CRC) vs. standard deviation (STD) curves showed that the process 1100 achieved the best performance regarding the bias-variance tradeoff. For testing on a clinical PET/CT dataset, the process 1100 achieved the highest CNR improvement ratio (53.35%±21.78%), as compared with the Gaussian (12.64%±6.15%, P=0.002), NLM guided by CT (24.35%±16.30%, P=0.002), BM4D (38.31%±20.26%, P=0.002), and Deep Decoder (41.67%±22.28%, P=0.002) methods. For testing on a clinical PET/MR dataset, the CNR improvement ratio of the proposed method achieved 46.80%±25.23%, higher than the Gaussian (18.16%±10.02%, P<0.0001), NLM guided by MR (25.36%±19.48%, P<0.0001), BM4D (37.02%±21.38%, P<0.0001), and Deep Decoder (30.03%±20.64%, P<0.0001) methods.

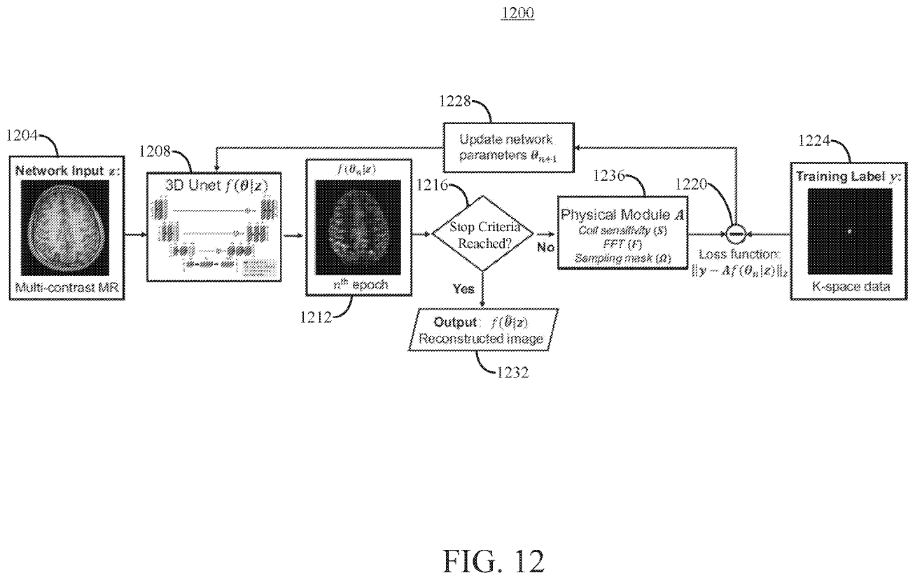

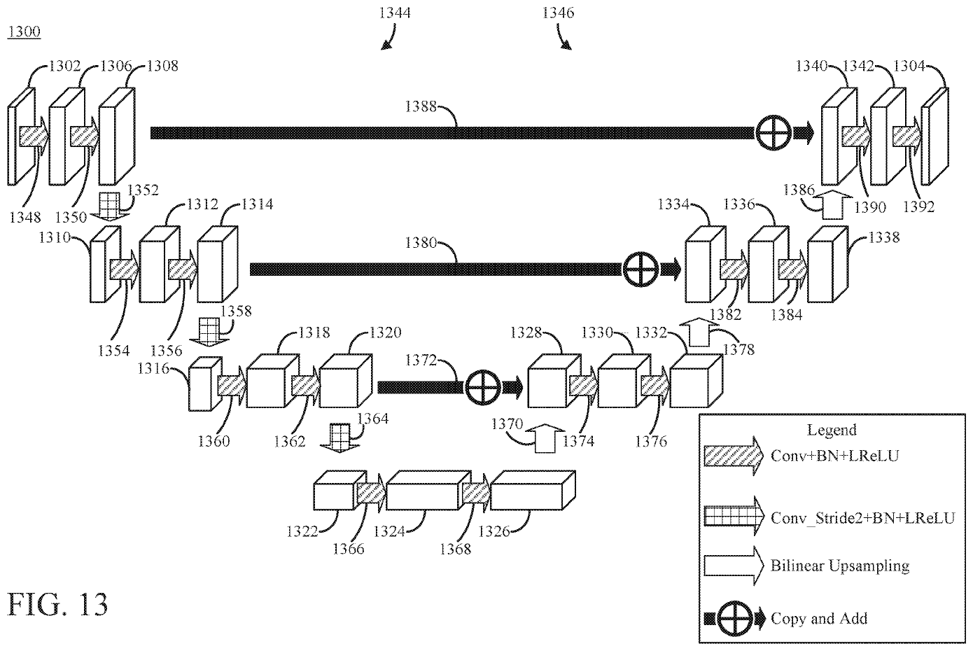

Referring now to FIG. 12, a flow 1200 for generating and/or reconstructing Arterial Spin Labeling (ASL) MR images is shown. The flow 1200 can include providing an input image 1204 to a model 1208. In some configurations, the input image 1204 can be a prior MR image of a patient. The model 1208 can generate and output a generated image 1212. In some configurations, the model 1208 can include a neural network, which will be described in detail below. In some configurations, the model 1208 can be a modified 3D U-net as shown in FIG. 13. In some configurations, the generated image 1212 can be a reconstructed image (e.g., a reconstructed ASL image) and/or a denoised image (e.g., a denoised ASL image).

The flow 1200 can include determining if stop criteria has been reached 1216. In some configurations, the flow 1200 can train the model 1208 for a predetermined number of epochs before determining that the stop criteria has been reached. For example, the stop criteria can be three hundred epochs. If the stop criteria has been reached (e.g., “NO” at 1216), the flow 1200 can proceed to loss function calculation 1220. In some configurations, the loss function can be an L2 loss function calculated between a training label 1224 and the generated image 1212. The flow 1200 can include calculating a loss function value based on a predetermined loss function . The flow 1200 can include calculating the loss function using the loss function value based on the loss function, the generated image 1212, a physical module 436(e.g., an identity matrix), and the training label 1224.

The flow can include updating model parameters 1228 of the model 1212. For example, the flow 1200 can adjust weights of the model 1208. Once the model has been trained using the same input image 1204 and the same training label 1224 until the stop criteria has been reached (e.g., at 1216), the flow 1200 can output the last generated image 1212 generated by the model 1208 as a final image 1232. In some configurations, the reconstructed image 1232 can be a reconstructed image and/or a denoised image. Specifics of the flow will be discussed below.

Specifically, an unsupervised deep learning approach for generating and/or reconstructing Arterial Spin Labeling (ASL) MR images is described. To train the network, T1-weighted anatomical image can be used as network input, with noisy ASL image as training label for denoising task or k-space data as training label for image reconstruction from sparsely sampled data application.

For MR inverse problems such as image denoising and reconstruction, the measured data, y∈ [symbol] ^{M×1}, can be written as

y=Ax+w (28)

where A∈ [symbol] ^{M×N} is the transformation matrix, x∈ [symbol] ^{N×1} is the unknown image to be estimated, w∈ [symbol] ^{M×1} is the noise, M is the size of measured data, and N is the number of pixels in image space. Supposing y is independent and identically distributed (i.i.d.) and the ith element of y, yᵢ, follows a distribution of p(yᵢ|x), x can be estimated based on the maximum likelihood framework as

$$\hat{x} = arg {max}_{x} log {\sum }_{i = 1}^{M} p ( {y}_{i} \vert x ) \qquad ( 29 )$$

The maximum-likelihood solution of equation (29) can be prone to noise and/or undersampling artifacts. The image x can be nonlinearly represented by a deep neural network as

x=f(θ|z) (30)

where f represents the neural network, θ denotes the unknown parameters of the neural network, and z denotes the prior image which is the input to the neural network. This representation can exploit multiple-contrast prior images, simply by increasing the number of network-input channels. With x substituted by the neural network representation in equation (30), the original data model in equation (28) becomes

y=Af(θ|z)+w (31)

where θ are the only unknown parameters to be determined. The task of recovering unknown image×from the measured data y is translated to finding an optimal set of network parameters θ that maximize the likelihood function:

$$\hat{θ} = arg {max}_{θ} log {\sum }_{i = 1}^{M} p ( {y}_{i} \vert θ ) \qquad ( 32 )$$

Once θ is determined, the reconstructed image \hat{x} is simply the output of the neural network, \hat{x}=(\hat{θ}|z).

### Image Denoising

For denoising applications, the transformation matrix 1236 becomes an identity matrix and y is the noisy image. The image noise w can be assumed to follow i.i.d. Gaussian distribution, and the maximum likelihood estimation in equation (32) can be explicitly written as

$$\hat{θ} = arg {min}_{θ} {‖ y - f ( θ \vert z ) ‖}_{2}^{2} \qquad ( 33 )$$

Optimization of equation (33) is a network training problem with L2 norm as the loss function. A unique advantage of this framework is that only the measured data from the subject is employed in the training process, with no additional training pairs needed. This can significantly reduce the training data size and thus allows using high-order optimization methods to train the network with better convergence properties. The L-BFGS algorithm can be used to solve equation (33), which is a Quasi-Newton method, using a history of updates to approximate the Hessian matrix. Compared to the commonly used first-order stochastic algorithms, L-BFGS is a monotonic algorithm and is preferred to solve equation (33) due to its stability and better performance as described above.

### Reconstruction from Sparsely Sampled Data

For image reconstruction, y is the measured k-space data and A is the forward model of imaging, which can be written as

A=ΩFS (34)

where Ω is the k-space sampling mask, [symbol] denotes Fourier transform, and S represents the coil sensitivities. Assuming i.i.d. Gaussian noise in the k-space data, the maximum likelihood estimation in equation (32) becomes

$$\hat{θ} = arg {min}_{θ} {‖ Ωℱ Sf ( θ \vert z ) - y ‖}_{2}^{2} \qquad ( 35 )$$

Directly training the neural network (estimating θ) using the loss function in equation (35) can be time consuming because the forward operator A and its adjoint is often computationally expensive and the neural network training needs more update steps compared to traditional iterative image reconstruction. Instead alternating direction method of multipliers (ADMM) was used to separate the reconstruction and network training steps. Another advantage is that this separation allows direct use of penalized image reconstruction methods at the image reconstruction step. One auxiliary variable×to convert (35) to the constrained format as below

min||Ω [symbol] Sx−y||₂²s.t.x=f(θ|z) (36)

The augmented Lagrangian format for the constrained optimization problem in (36) is:

$${L}_{ρ} = {‖ Ωℱ Sx - y ‖}_{2}^{2} + {y}^{T} ( x - f ( θ \vert z ) ) + \frac{ρ}{2} {‖ x - f ( θ \vert z ) ‖}_{2}^{2} \qquad ( 37 )$$

where y is the dual variable and ρ is the penalty parameter. It can be solved by the ADMM algorithm iteratively in three steps

$${θ}^{n + 1} = {argmin}_{θ} {‖ f ( θ \vert z ) - ( {x}^{n} + {μ}^{n} ) ‖}_{2}^{2} \qquad ( 38 )$$

$${x}^{n + 1} = {argmin}_{x} {‖ Ωℱ Sx - y ‖}_{2}^{2} + \frac{ρ}{2} {‖ x - f ( {θ}^{n + 1} \vert z ) + {μ}^{n} ‖}_{2}^{2} \qquad ( 39 )$$

$${μ}^{n + 1} = {μ}^{n} + {x}^{n + 1} - f ( {θ}^{n + 1} \vert z ) \qquad ( 40 )$$

where μ=(1/ρ)y is the scaled dual variable. With the ADMM algorithm, the original constrained optimization problem is decoupled into a network training problem (38) and a penalized reconstruction problem (39). At each step of iteration, the parameters of the neural network (θⁿ⁺¹ are estimated by minimizing the L2 norm difference between the output of the network (f(θ|z) and the training label (xⁿ+μⁿ). The training label are updated consecutively by solving subproblem (39) and (40), respectively. The L-BFGS algorithm can be used to solve subproblem (38). By setting the first order derivative to zero, the normal equation for subproblem (39) can be expressed as

[(Ω [symbol] S)^{H}ΩFS+ρI]x=(Ω [symbol] S)^{H}y+ρ[f(θⁿ⁺¹|z)−μⁿ] (41)

The reconditioned conjugate gradient (PCG) algorithm can used to solve the above normal equation (41).

Referring now to FIG. 13, an exemplary neural network 1300 is shown. In some configurations, the neural network 1300 can include at least a portion of the same components as the neural network 1300 in FIG. 13. In some configurations, the neural network 1300 can include a left-side encoder path 1344 and a right-side decoder path 1346.

In some configurations, the neural network 1300 can receive an input image 1302. In some configurations, the input image 1302 can be a single channel three dimensional image (e.g., an MR image of a patient). In some configurations, the neural network 1300 can generate and output a predicted image 1304 (e.g., a denoised ASL image). The predicted image 1304 can be a multi-channel three dimensional image (e.g., a two channel image). For an ASL image, the two channels can represent the real and imaginary parts of the ASL image. In some configurations, the neural network 1300 can include a number of feature maps coupled together by a combination of different layers. In some configurations, the neural network 1300 can include a first feature map 1306, a second feature map 1308, a third feature map 1310, a fourth feature map 1312, a fifth feature map 1314, a sixth feature map 1316, a seventh feature map 1318, an eighth feature map 1320, a ninth feature map 1322, a tenth feature map 1324, a eleventh feature map 1326, a twelfth feature map 1328, a thirteenth feature map 1330, a fourteenth feature map 1332, a fifteenth feature map 1334, a sixteenth feature map 1336, a seventeenth feature map 1338, an eighteenth feature map 1340, and a nineteenth feature map 1342.

In some configurations, the feature maps 1306-1342 can be the same as the feature maps 506-542 in the neural network 500 with a few differences. In some configurations, the first feature map 1306 can include twelve channels, the second feature map 1308 can include twelve channels, the fourth feature map 1312 can include twenty-four channels, the fifth feature map 1314 can include twenty-four channels, the seventh feature map 1318 can include forty-eight channels, the eighth feature map 1320 can include forty-eight channels, the tenth feature map 1324 can include ninety-six channels, the eleventh feature map 1326 can include ninety-six channels, the twelfth feature map 1328 can include forty-eight channels, the thirteenth feature map 1330 can include forty-eight channels, the fifteenth feature map 1334 can include twenty-four channels, the sixteenth feature map 1336 can include twenty-four channels, the eighteenth feature map 1340 can include twelve channels. In some configurations, the nineteenth feature map 1342 can include twice as many channels as the eighteenth feature map 1340 (e.g., twenty-four channels) to generate the two-channel predicted image 1304.

As described above, the feature maps 1306-1342 can be coupled together using a number of different layers. The layers may be arranged into groups of common layer arrangements. For example, a layer group can include a convolutional layer (e.g., a convolutional layer with a stride length of 1), a BN layer, and an LReLU layer. In some configurations, the neural network 1300 can include a first layer group 1348, a second layer group 1350, a third layer group 1352, a fourth layer group 1354, a fifth layer group 1356, a sixth layer group 1358, a seventh layer group 1360, an eighth layer group 1362, a ninth layer group 1364, a tenth layer group 1366, a eleventh layer group 1368, a twelfth layer group 1370, a thirteenth layer group 1372, a fourteenth layer group 1374, a fifteenth layer group 1376, a sixteenth layer group 1378, a seventeenth layer group 1380, an eighteenth layer group 1382, a nineteenth layer group 1384, a twentieth layer group 1386, a twenty-first layer group 1388, a twenty-second layer group 1390, and a twenty-third layer group 1392. In some configurations, the layer groups 1348-1392 can be include the same layer arrangements as the layer groups 548-592 in the neural network 500.

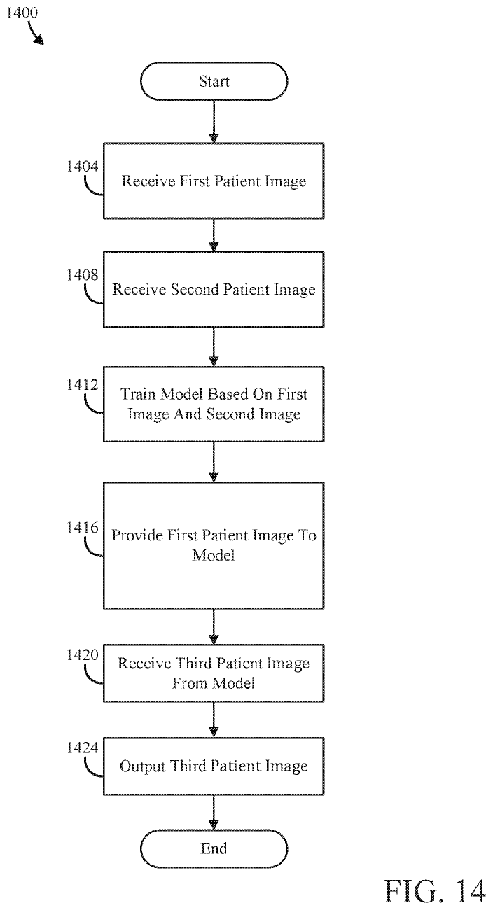

Referring now to FIG. 14, yet another exemplary process 1400 for training a model is shown. In some configurations, the model can include the neural network 1300 in FIG. 13. In some configurations, the process 1400 can train the model to generate a denoised ASL image and/or a reconstructed ASL image using an MR image. In some configurations, the medical image analysis application 132 can include the process 1400. In some configurations, the processes 1400 can be implemented as computer readable instructions on a memory or other storage medium and executed by a processor. In some configurations, the model can be a neural network without any pretraining. For example, the model can include randomized values for all trainable parameters.

At 1404, the process 1400 can receive a first patient image. In some configurations, the first patient image can be an MR image associated with the patient. In some configurations, the first patient image can be a T1-weighted multi-contrast MR image. In some configurations, the first patient image can be the input image 1204 in FIG. 12. In some configurations, the first patient image can be a single channel image having pixel intensities normalized to a range of [0, 1].

At 1408, the process 1400 can receive a second patient image. In some configurations, the second patient image can be a noisy ASL image associated with the patient. In some configurations, the noisy PET image can be sparsely sampled data (e.g., k-space data). In some configurations, the second patient image can be previously acquired concurrently and/or simultaneously along with the first patient image.

At 1412, the process 1400 can train the model based on the first patient image and the second patient image. As described above, in some configurations, the model can include the neural network 1300 in FIG. 13. In some configurations, the process 1400 can provide the first patient image to the model (e.g., as the input image 1302), receive an output image from the model (e.g., the predicted image 1304). Based on the output image and the second patient image, the process 1400 can update weights of parameters in the model.

In configurations where the process 1400 trains the model to denoise an image, the first patient image can be a noisy ASL image. In some configurations, the process 1300 can iteratively provide the first patient image to the model, receive an output image from the model, and update the model parameters based on the output image and the noisy ASL image (e.g., the second patient image). In some configurations, the process 1400 can update the model by solving equation (33) using L-BFGS as described above.

In configurations where the process 1400 trains the model to reconstruct an image, the first patient image can be sparsely sampled data (e.g., k-space data). In some configurations, the process 1300 can iteratively provide the first patient image to the model, receive an output image from the model, and update the model parameters based on the output image and the sparsely sampled data (e.g., the second patient image). In some configurations, the process 1400 can update the model by solving equations (38)-(40) as described above.

At 1416, the process 1400 can provide the first patient image to the model. At 1416, the model is trained and can be referred to as a trained model.

At 1420, the process 1400 can receive a third patient image from the model. The third patient image may be an image associated with an imaging modality that differs from the first patient image or the second patient image. The third patient image can be a denoised ASL image if the second patient image was a noisy ASL image. The third patient image can be a reconstructed ASL image if the second patient image was sparsely sampled data.

At 1424, the process 1400 can output the third patient image to at least one of a memory or a display. In some configurations, at 1424, the process 1400 can cause the third patient image to be displayed on a display (e.g., the display 108). In some configurations, at 1424, the process 1400 can generate a report based on the third patient image. In some configurations, at 1424, the process 1400 can output the report to at least one of a memory or a display. In some configurations, at 1424, the process 1400 can cause the report to be displayed on a display (e.g., the display 108). It is understood that at least some of the steps included in the process 1400 can be rearranged and/or executed in parallel.

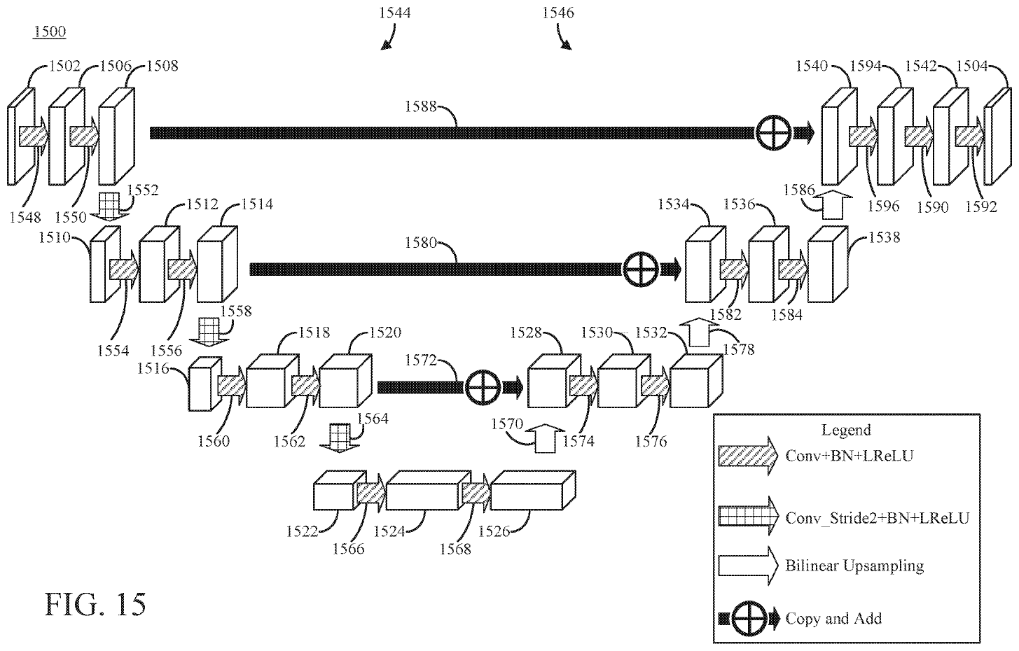

Referring now to FIG. 15, an exemplary neural network 1500 is shown. In some configurations, the neural network 1500 can include at least a portion of the same components as the neural network 1500 in FIG. 15. In some configurations, the neural network 1500 can include a left-side encoder path 1544 and a right-side decoder path 1546. In some configurations, the neural network 1500 can receive an input image 1502. In some configurations, the input image 1502 can be a single channel three dimensional image (e.g., a summation of low energy and high energy CT images reconstructed using filtered back projection (FBP)). In some configurations, the neural network 1500 can generate and output a predicted image 1504 (e.g., a reconstructed low-dose dual energy CT (DECT) image). In some configurations, the predicted image 1504 can be a multi-channel three dimensional image (e.g., a two channel image). For a DECT image, the two channels can represent a low energy image (denoted as xₗ) and a difference image (denoted as x_{d}).

In some configurations, the neural network 1500 can include a number of feature maps coupled together by a combination of different layers. In some configurations, the neural network 1500 can include a first feature map 1506, a second feature map 1508, a third feature map 1510, a fourth feature map 1512, a fifth feature map 1514, a sixth feature map 1516, a seventh feature map 1518, an eighth feature map 1520, a ninth feature map 1522, a tenth feature map 1524, a eleventh feature map 1526, a twelfth feature map 1528, a thirteenth feature map 1530, a fourteenth feature map 1532, a fifteenth feature map 1534, a sixteenth feature map 1536, a seventeenth feature map 1538, an eighteenth feature map 1540, and a nineteenth feature map 1542.

As described above, the feature maps 1506-1542 can be coupled together using a number of different layers. The layers may be arranged into groups of common layer arrangements. For example, a layer group can include a convolutional layer (e.g., a convolutional layer with a stride length of 1), a BN layer, and an LReLU layer. In some configurations, the neural network 1500 can include a first layer group 1548, a second layer group 1550, a third layer group 1552, a fourth layer group 1554, a fifth layer group 1556, a sixth layer group 1558, a seventh layer group 1560, an eighth layer group 1562, a ninth layer group 1564, a tenth layer group 1566, a eleventh layer group 1568, a twelfth layer group 1570, a thirteenth layer group 1572, a fourteenth layer group 1574, a fifteenth layer group 1576, a sixteenth layer group 1578, a seventeenth layer group 1580, an eighteenth layer group 1582, a nineteenth layer group 1584, a twentieth layer group 1586, a twenty-first layer group 1588, a twenty-second layer group 1590, and a twenty-third layer group 1592.

In some configurations, the feature maps 1506-1542 can be the same as the feature maps 506-542 in the neural network 500 with a few differences. In some configurations, the neural network 1500 can include a non-local layer 1594 coupled to the eighteenth feature map 1540 by a twenty-fourth layer group 1596 including a convolutional layer (e.g., a convolutional layer with a stride length of 1), a BN layer, and an LReLU layer. The non-local layer 1594 can also be coupled to the nineteenth feature map 1542 by the twenty-third layer group 1592. In some configurations, the nineteenth feature map 1542 can include twice as many channels as the eighteenth feature map 1540 (e.g., twenty-four channels) to generate the two-channel predicted image 1504.

For DECT scans, sinogram data yₗ from low energy (typically 80 kVp), and yₕ from high energy (typically 140 kVp) are acquired simultaneously. Traditionally, the low energy image xₗ and high energy image xₕ are reconstructed from yₗ and yₕ independently based on the Poisson model,

$$L ( y \vert x ) ∝ {\sum }_{i = 1}^{M} {y}_{i} log ( {b}_{i} {e}^{- {[ Ax ]}_{i}} + {r}_{i} ) - {b}_{i} {e}^{- {[ Ax ]}_{i}} - {r}_{i} \qquad ( 42 )$$

where A is the system matrix, bᵢ denotes the blank scan for detector i, and rᵢ is the mean value of background noise for detector i. xₗ is noisier than xₕ due to higher attenuation coefficients at 80 kVp compared to 140 kVp. For clinical diagnosis, the difference image x_{d}=xₗ−xₕ is important as it contains the injected contrast agent information, such as iodine concentration. Always, x_{d} is obtained after reconstructing xₗ and x_{d} independently, which is noisy in low-dose scenarios. Various methods have been proposed for DECT image reconstruction.

Supervised deep learning approaches have been applied to medical image reconstruction, but these approaches require a large number of high-quality training images with k-space data or sinograms also available, which are difficult to acquire or obtain. The deep image prior (DIP) framework shows that convolutional neural networks (CNNs) can learn intrinsic structural information from the corrupted images. In DIP framework, random noise is used as the network input and no high-quality training labels are needed. Furthermore, it has been shown that when the network input is not random noise but high-quality prior image from the same subject, the denoising results can be further improved. The nosier images, xₗ and x_{d}, can be represented as the two-channel output from a neural network as

xₗ=f(θ|α)₁,x_{d}=f(θ|α)₂ (43)

where f represents the neural network (e.g., the neural network 1500), θ stands for the network trainable parameters, α is the network input, and the subscript of f( ) implies the output channel number. Here xₗ+xₕ is employed as the network input α as it has lower noise but preserves all image structures. To further utilize the prior information, the non-local layer 1594 can function as additional constraint. The non-local layer 1594 can be pre-calculated based on xₗ+xₕ using a Gaussian weighting function. After substituting xₗ, xₕ=xₗ−x_{d} with the neural network representation (43) and expressing the objective function in a constrained format, the original dual-energy CT reconstruction model can be rewritten as

max L( [symbol] ₗ|xₗ)+L( [symbol] ₕ|xₕ)

s.t.xₗ=f(θ|α)₁, xₕ=f(θ|α)₁+f(θ|α)₂ (44)

which can be solved by the ADMM algorithm iteratively in three steps. The first step is

$${θ}^{n + 1} = {argmin}_{θ} {‖ {f ( θ \vert α )}_{1} - ( {x}_{l}^{n} + {μ}_{l}^{n} ) ‖}^{2} + {‖ {f ( θ \vert α )}_{2} - ( {x}_{l}^{n} - {x}_{h}^{n} + {μ}_{l}^{n} - {μ}_{h}^{n} ) ‖}^{2} \qquad ( 45 )$$

which is a network training problem and can be solved using the L-BFGS algorithm due to its monotonic property. The second step is

$${x}_{l}^{n + 1} , {x}_{h}^{n + 1} = {argmax}_{{x}_{l} , {x}_{h}} L ( {y}_{l} \vert {x}_{l} ) + L ( {y}_{h} \vert {x}_{h} ) - \frac{ρ}{2} {‖ {x}_{l} - {f ( {θ}^{n + 1} \vert α )}_{1} + {μ}_{l}^{n} ‖}^{2} - \frac{ρ}{2} {‖ {x}_{h} - {f ( {θ}^{n + 1} \vert α )}_{1} - {f ( {θ}^{n + 1} \vert α )}_{2} + {μ}_{h}^{n} ‖}^{2} \qquad ( 46 )$$

which is a penalized CT reconstruction, and can be solved using the SQS algorithm. Note that xₗ and xₕ can be updated independently in this step. The third step is the update of dual variables μₗ and μₕ.

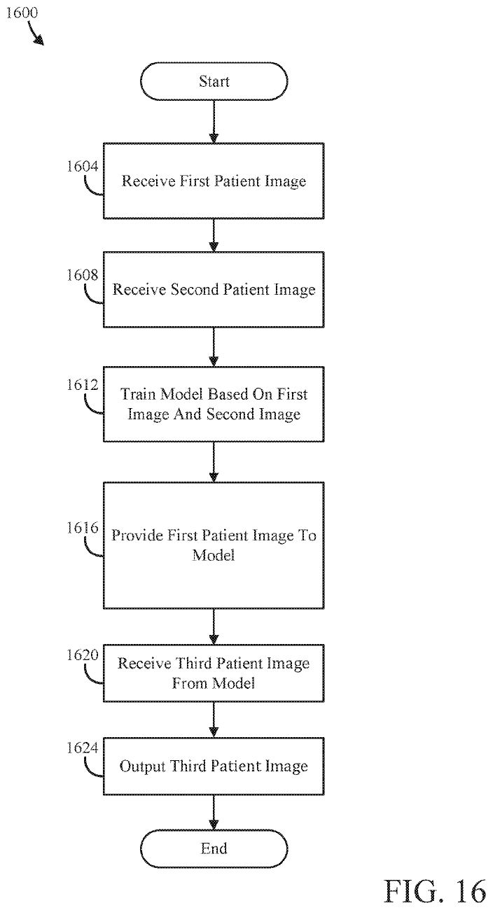

Referring now to FIG. 16, still yet another exemplary process 1600 for training a model is shown. In some configurations, the model can include the neural network 1500 in FIG. 15. In some configurations, the process 1600 can train the model to generate a reconstructed DECT image using an MR image. In some configurations, the medical image analysis application 132 can include the process 1600. In some configurations, the process 1600 can be implemented as computer readable instructions on a memory or other storage medium and executed by a processor. In some configurations, the model can be a neural network without any pretraining. For example, the model can include randomized values for all trainable parameters.

At 1604, the process 1600 can receive a first patient image. In some configurations, the first patient image can be a single channel three dimensional image CT image. The single channel three dimensional CT image can be a summation of low energy and high energy CT images reconstructed using filtered back projection (FBP)). In some configurations, the first patient image can be a single channel image having pixel intensities normalized to a range of [0, 1].

At 1608, the process 1600 can receive a second patient image. In some configurations, the second patient image can be a two channel three dimensional DECT image. The DECT image can include a low energy image (e.g., as a first channel) and a difference image (e.g., as a second channel). In some configurations, the second patient image can be previously acquired concurrently and/or simultaneously along with the first patient image.

At 1612, the process 1600 can train the model based on the first patient image and the second patient image. As described above, in some configurations, the model can include the neural network 1500 in FIG. 15. In some configurations, the process 1600 can provide the first patient image to the model (e.g., as the input image 1502), receive an output image from the model (e.g., the predicted image 1504). Based on the output image and the second patient image, the process 1600 can update weights of parameters in the model. In some configurations, the process 1600 can update the model by solving equations (45)-(46) and updating μₗ and μₕ as described above.

At 1616, the process 1600 can provide the first patient image to the model. At 1616, the model is trained and can be referred to as a trained model. At 1620, the process 1600 can receive a third patient image from the model. The third patient image can be a reconstructed DECT image. The reconstructed DECT image can include a low energy image (e.g., as a first channel) and a difference image (e.g., as a second channel).

At 1624, the process 1600 can output the third patient image to at least one of a memory or a display. In some configurations, at 1624, the process 1600 can cause the third patient image to be displayed on a display (e.g., the display 108). In some configurations, at 1624, the process 1600 can generate a report based on the third patient image. In some configurations, at 1624, the process 1600 can output the report to at least one of a memory or a display. In some configurations, at 1624, the process 1600 can cause the report to be displayed on a display (e.g., the display 108). It is understood that at least some of the steps included in the process 1600 can be rearranged and/or executed in parallel.

Referring back to FIG. 5, in some configurations, the model neural network 500 can be trained to generate a parametric PET image (e.g. BP parametric PET image) based on a CT image and a reconstructed PET image of a patient. Logan graphical method has been used to analyze reversibly binding neuroreceptor tracers. A reformulation of the Logan model, which uses a reference region to replace arterial blood sampling, can be expressed as

$$\frac{{\int }_{0}^{T} {C}_{ROI} ( t ) dt}{{C}_{ROI} ( t )} = DVR \frac{{\int }_{0}^{T} {C}_{ref} ( t ) dt}{{C}_{ROI} ( t )} + V ( t > {t}^{*} ) \qquad ( 47 )$$

where C_{ROI}(t) and C_{ref}(t) are the radioactivity concentrations in ROI and reference tissue at time t, respectively. After equilibrium time t_, the plot becomes linear. The slope of the linear part is distribution volume ratio (DVR), which has a relationship with binding potential (BP) by BP=DVR−1. In testing, reference tissue was drawn in the muscle region. For convenience, yₘ∈ [symbol] ^{N×1} and xₘ∈ [symbol] ^{N×1} are used to represent

$$\frac{{\int }_{0}^{T} {C}_{ROI} ( t ) dt}{{C}_{ROI} ( t )} and \frac{{\int }_{0}^{T} {C}_{ref} ( t ) dt}{{C}_{ROI} ( t )}$$

in the mth frame in the rest of the paper. The optimization problem for parametric image reconstruction can be written as

$${min}_{K} Ψ ( K ) = {min}_{K} \frac{1}{2} {‖ Y - PK ‖}^{2} \qquad ( 48 )$$

where Y∈ [symbol] ^{I×1}={yₘ|m>m*}, P∈ [symbol] ^{I×2N}={pₘ|pₘ=[diag(xₘ), I_{N}], m>m*}, and K∈ [symbol] ^{2N×1}=[DV R′, V′]′. m* stands for the equilibrium frame and I_{N} is a N×N identity matrix. Inspired by the deep image prior framework which shows that CNN can learn intrinsic structure information from the corrupted image and its related works, a constraint is introduced by representing the unknown parametric image DV R by

DV R=f(θ|z) (49)

where f represents the neural network, θ are the unknown parameters of the neural network, z denotes the neural network input (e.g., the input image 502) which comes from CT images of the same patient. With this constraint, the optimization problem for parametric image reconstruction can be written as

minimize ½||Y−PK||²

subject to DV R=f(θ|z) (50)

The augmented Lagrangian format of (50) can be represented as

$${L}_{ρ} = \frac{1}{2} {‖ Y - PK ‖}^{2} + \frac{ρ}{2} {‖ DVR - f ( θ \vert z ) + μ ‖}^{2} - \frac{ρ}{2} {‖ μ ‖}^{2} \qquad ( 51 )$$

The augmented Lagrangian optimization problem in (51) can be solved by ADMM algorithm in three steps:

$${θ}^{n + 1} = {argmin}_{θ} {‖ f ( θ \vert z ) - ( {DVR}^{n} + {μ}^{n} ) ‖}^{2} \qquad ( 52 )$$

$${DVR}^{n + 1} = {argmin}_{DVR} \frac{1}{2} {‖ Y - PK ‖}^{2} + \frac{ρ}{2} {‖ DVR - f ( {θ}^{n + 1} \vert z ) + {μ}^{n} ‖}^{2} \qquad ( 53 )$$

$${μ}^{n + 1} = {μ}^{n} + {DVR}^{n + 1} - f ( {θ}^{n + 1} \vert z ) \qquad ( 54 )$$

The subproblem for θ in (52) is a network training problem with L2 norm as loss function. The network structure employed in this problem can be the neural network 500 in FIG. 5. The total number of unknown parameters for the neural network 500 can be about 1.5 million. To reduce the fluctuations caused by small batches, the whole 3D volume was employed as the network input and the L-BFGS method was adopted to train the network due to its stability and better performance observed in the experiments. Optimization transfer can be used to solve subproblem (53). The ith element of the data-fitting part Ψ(K) in the subproblem (53) can be decoupled by

$${Ψ}_{i}^{n} ( {P}_{i} K ) \leq {\sum }_{j = 1}^{2 N} \frac{{P}_{ij} {K}_{j}^{n}}{{P}_{i} {K}^{n}} {Ψ}_{i}^{n} ( \frac{{P}_{i} {K}^{n}}{{K}_{j}^{n}} {K}_{j} ) \qquad ( 55 )$$

Thus, subproblem (53) has the following surrogate function

$${K}_{j}^{n + 1} arg {min}_{{K}_{j}} {\sum }_{i = 1}^{1} {\sum }_{j = 1}^{2 N} \frac{1}{2} \frac{{P}_{ij} {K}_{j}^{n}}{{P}_{i} {K}^{n}} {( {Y}_{i} - \frac{{P}_{i} {K}^{n}}{{K}_{j}^{n}} {K}_{j} )}^{2} + {\sum }_{j = 1}^{2 N} \frac{{P}_{j}}{2} {( {K}_{j} - {F}_{j}^{n + 1} + {U}_{j}^{n} )}^{2} \qquad ( 56 )$$

$${ρ}_{j} = \{ \begin{matrix}ρ & j = 1 , … , N , \\ 0 & j = N + 1 , … , 2 N ,\end{matrix}$$

In the surrogate function (56), the penalty term in the subproblem (53) is written as a function of Kⱼ by using a conditional parameter ρⱼ. f(θⁿ⁺¹|z) and μⁿ are replaced by Fⁿ⁺¹∈ [symbol] ^{2N×1}=[f′(θⁿ⁺¹|z),0′]′ and Uⁿ∈ [symbol] ^{2N×1}=[μⁿ′, 0′]′, respectively. The resulting iterative update equation for Kⱼ is

$${K}_{j}^{n , m + 1} = {K}_{j}^{n , m} \frac{{\sum }_{i = 1}^{1} {P}_{ij} {Y}_{i} + {ρ}_{j} ( {F}_{j}^{n + 1} - {U}_{j}^{n + 1} )}{{\sum }_{i = 1}^{1} {P}_{ij} ( {P}_{i} {K}^{n , m} ) + {ρ}_{j} {K}_{j}^{n , m}} \qquad ( 57 )$$

where m=1, . . . , M+1 is the number of sub-iterations. DV Rⱼⁿ⁺¹ is equal to [Kⱼ^{m,M+1]+(j=}1, . . . ,N). In some configurations, to solve equations (52)-(54), equation (52) can be solved over ten iterations, and equations (53) can be solved over two iterations.

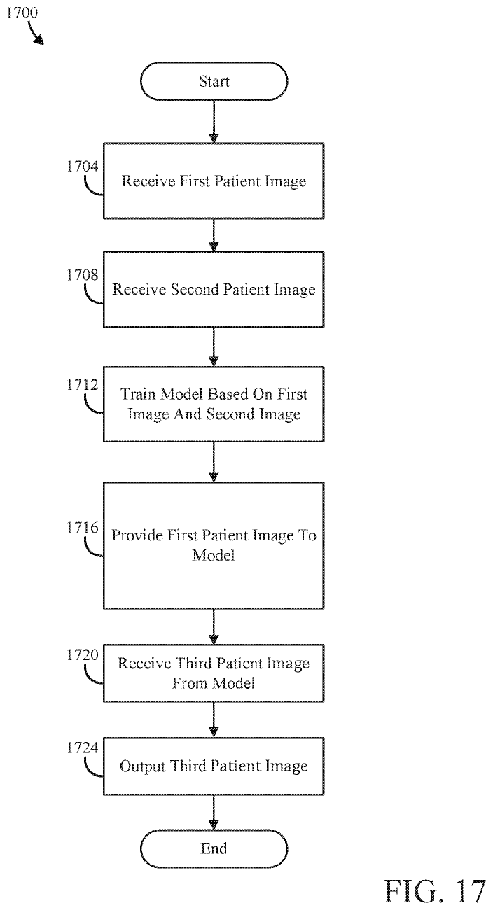

Referring now to FIG. 17, an additional exemplary process 1700 for training a model is shown. In some configurations, the model can include the neural network 1500 in FIG. 15. In some configurations, the process 1700 can train the model to generate a parametric PCT image using a CT image. In some configurations, the medical image analysis application 132 can include the process 1700. In some configurations, the process 1700 can be implemented as computer readable instructions on a memory or other storage medium and executed by a processor. In some configurations, the model can be a neural network without any pretraining. For example, the model can include randomized values for all trainable parameters.

At 1704, the process 1700 can receive a first patient image. In some configurations, the first patient image can be a single channel three dimensional image CT image. The single channel three dimensional CT image can be a low-dose CT image. In some configurations, the first patient image can be a single channel image having pixel intensities normalized to a range of [0, 1].

At 1708, the process 1700 can receive a second patient image. In some configurations, the second patient image can be a reconstructed PET image. In some configurations, the reconstructed PET image can be reconstructed using 3-dimensional ordered subset expectation maximization (3D-OSEM). For example, PET images can be acquired with a multi-bed-position dynamic scan and reconstructed using 3D-OSEM with three iterations and twenty-one subsets.

At 1712, the process 1700 can train the model based on the first patient image and the second patient image. As described above, in some configurations, the model can include the neural network 500 in FIG. 5. In some configurations, the process 1700 can provide the first patient image to the model (e.g., as the input image 502), receive an output image from the model (e.g., the predicted image 504). Based on the output image and the second patient image, the process 1700 can update weights of parameters in the model. In some configurations, the process 1700 can update the model by solving equations (52)-(53) and updating μₗ and μₕ as described above.

At 1716, the process 1700 can provide the first patient image to the model. At 1716, the model is trained and can be referred to as a trained model. At 1720, the process 1700 can receive a third patient image from the model. The third patient image can be a parametric PET image. The parametric PET image can include a low energy image (e.g., as a first channel) and a difference image (e.g., as a second channel).

At 1724, the process 1700 can output the third patient image to at least one of a memory or a display. In some configurations, at 1724, the process 1700 can cause the third patient image to be displayed on a display (e.g., the display 108). In some configurations, at 1724, the process 1700 can generate a report based on the third patient image. In some configurations, at 1724, the process 1700 can output the report to at least one of a memory or a display. In some configurations, at 1724, the process 1700 can cause the report to be displayed on a display (e.g., the display 108). It is understood that at least some of the steps included in the process 1700 can be rearranged and/or executed in parallel.

The present invention has been described in terms of one or more preferred configurations, and it should be appreciated that many equivalents, alternatives, variations, and modifications, aside from those expressly stated, are possible and within the scope of the invention.

1. An image generation system comprising:
at least one processor; and
at least one non-transitory, computer-readable memory accessible by the processor and having instructions that, when executed by the processor, cause the processor to:
receive a first patient image associated with a patient;
receive a second patient image associated with the patient;
train an untrained model based on the first patient image and the second patient image;
provide the first patient image to the model;
receive a third patient image from the model; and
output the third patient image to at least one of a storage system or a display.

2. The system of claim 1, wherein the model comprises a neural network comprising a number of feature maps, wherein at least a portion of the feature maps are downsampled by a 3×3×3 three dimensional convolutional layer having a stride of 2×2×2.

3. The system of claim 1, wherein the model comprises a fully convolutional neural network, and wherein the fully convolutional neural network does not include a pooling layer.

4. The system of claim 1, wherein the model comprises a neural network comprising an encoder path and a decoder path, the neural network further comprising a number of identity mapping layers comprising skip connections, the identity mapping layers coupled between the encoder path and a decoder path.

5. The system of claim 1, wherein the first patient image is a magnetic resonance image, the second patient image is a raw sinogram, and the third patient image is a reconstructed positron emission tomography image.

6. The system of claim 1, wherein the system trains the untrained model with training data only comprising the first patient image and the second patient image.

7. The system of claim 1, wherein the system is coupled to an imaging system, and wherein the system is further configured receive the first patient image from the imaging system and receive the second patient image from the imaging system.

8. The system of claim 1, wherein the first patient image is a magnetic resonance image, the second patient image is a noisy positron emission tomography image, and the third patient image is a denoised positron emission tomography image.

9. The system of claim 1, wherein the system is configured to train the untrained model by:
solving an objection function for a predetermined number of epochs based on the first patient image and the second patient image, wherein for each epoch the system is configured to:
solve a first subproblem of the objection function for a first predetermined number of iterations; and
solve a second subproblem of the objection function for a first predetermined number of iterations to update the model using the limited memory Broyden-Fletcher-Goldfarb-Shanno algorithm.

10. The system of claim 1, wherein the first subproblem is solved based on a Poisson distribution.

11. The system of claim 1, wherein the system is further configured to generate a report based on the third patient image and output the report to a display.

12. An image generation method comprising:
receiving a first patient image associated with a patient;
receiving a second patient image associated with the patient;
training an untrained model based on the first patient image and the second patient image;
providing the first patient image to the model
receiving a third patient image from the model; and
outputting the third patient image to at least one of a storage system or a display.

13. The method of claim 12, wherein the model comprises a neural network comprising a number of feature maps, wherein at least a portion of the feature maps are downsampled by a 3×3×3 three dimensional convolutional layer having a stride of 2×2×2.

14. The method of claim 12, wherein the model comprises a fully convolutional neural network, and wherein the fully convolutional neural network does not include a pooling layer.

15. The method of claim 12, wherein the model comprises a neural network comprising an encoder path and a decoder path, the neural network further comprising a number of identity mapping layers comprising skip connections, the identity mapping layers coupled between the encoder path and a decoder path.

16. The method of claim 12, wherein the first patient image is a magnetic resonance image, the second patient image is a raw sinogram, and the third patient image is a reconstructed positron emission tomography image.

17. The method of claim 12, wherein the training the untrained model comprises training the model only using the first patient image and the second patient image.

18. The method of claim 12, wherein the first patient image is a magnetic resonance image, the second patient image is a noisy positron emission tomography image, and the third patient image is a denoised positron emission tomography image.

19. The method of claim 12, wherein the training the untrained model comprises solving an objection function for a predetermined number of epochs based on the first patient image and the second patient image, wherein for each epoch the method comprises:
solving a first subproblem of the objection function for a first predetermined number of iterations; and
solving a second subproblem of the objection function for a first predetermined number of iterations to update the model using the limited memory Broyden-Fletcher-Goldfarb-Shanno algorithm.

20. The method of claim 12, wherein the first subproblem is solved based on a Poisson distribution.

21. The method of claim 12, wherein at least two of the first patient image, the second patient image, and the third patient image are images produced by different medical imaging modalities.
